(19) United States
(12) Patent Application Publication
(10) Pub. No.: US 2026/0069437 A1
(43) Pub. Date: Mar. 12, 2026

(54) SYSTEM AND METHOD FOR IMPROVED ATTACHMENT OF ASSISTIVE DEVICES

(71) Applicants: Nicholas Brmthal, Oakland, CA (US); Alexandra Stavrakis, Oakland, CA (US); Will Flanagan, Oakland, CA (US); Haley Warren, Oakland, CA (US); THE REGENTS OF THE UNIVERSITY OF CALIFORNIA, Oakland, CA (US)

(72) Inventors: Tyler Clites, Calabasas, CA (US); Nicholas Bernthal, Oakland, CA (US); Alexandra Stavrakis, Oakland, CA (US); Will Flanagan, Oakland, CA (US); Haley Warren, Oakland, CA (US)

(21) Appl. No.: 19/107,268

(22) PCT Filed: Aug. 31, 2023
(86) PCT No.: PCT/US2023/031684

§ 371 Date: Feb. 27, 2025

Related U.S. Application Data
(60) Provisional application No. 63/374,016, filed on Aug. 31, 2022.

(51) Int. Cl. A61F 2/80; A61F 2/68; A61F 2/70; A61F 2/78

(52) U.S. Cl. CPC A61F 2/80; A61F 2/70; A61F 2/7812; A61F 2002/6863; A61F 2002/701

(57) ABSTRACT

Disclosed is a magnetic prosthetic suspension system. The system includes a magnetic implant configured to be fixedly attached to bone at a target site of a subject, a socket configured to fit over the target site of the subject, and a magnet positioned on or within the socket, where the magnet is configured to generate a magnetic attractive force between the magnetic implant and the magnet that suspends the socket at the target site of the subject.

### CROSS REFERENCE TO RELATED APPLICATIONS

This application claims priority to U.S. provisional application Ser. No. 63/374,016 filed on Aug. 31, 2022, the contents of which are incorporated herein by reference in its entirety.

### BACKGROUND OF THE INVENTION

Paramount among the challenges faced by persons with amputation are the pain, discomfort, and potential tissue damage associated with prolonged prosthetic socket use. These challenges affect the overwhelming majority of patients, such that the primary patient-reported problems for both lower-and upper-extremity amputation are related to deficiencies of the conventional socket. In addition to reduction in quality of life, ill-fitting sockets pose a substantial healthcare burden: each patient visits their prosthetist an average of 7 times per year, with 68% of visits related to socket suspension. The average cost of prosthesis-related care for a patient with above-knee amputation is $4.5k per year. Extrapolated across the estimated 370,000 people living with above-knee amputation in the US, this corresponds to over $1.5 billion spent annually on prosthetic management. These costs are driven largely by regular fittings and adjustments, which are necessary to accommodate atrophy and volume changes in the residual limb.

One of the greatest obstacles posed by sockets is their reliance on residual soft tissues for suspension (FIG. 1A). Conventional suspension systems, which typically use either suction or pin-lock mechanisms, hang the entirety of the artificial limb from the skin, fat, and muscles of the residual limb. This causes discomfort anytime the prosthetic foot is not on the ground (e.g. the swing phase of gait) and can lead to pistoning, which is the name given to vertical movement of the residual limb within the socket. Pistoning is painful and can be devastating to long-term residuum health, because the internal amputated bone forcefully compresses the tissues of the distal residual limb with every step. This sustained repetitive trauma is damaging to all tissues of the distal residuum, leading to irritation and ulceration of the skin, bursitis and increased scar formation in the muscle and tendon, neuroma pain in the nerves, bruising and heterotopic ossification in the bones, and even arthritis in adjacent joints. Highlighting the pervasiveness of these issues, a 2009 survey showed that 68% of people with lower-extremity amputation experienced at least one skin lesion in the month prior to completing the survey; this regularly impacts all patients with amputation in a major way.

Unfortunately, some degree of pistoning is inevitable with all conventional suspension systems, due to regular fluctuations in residual limb volume. Conventional systems rely on snug fit between socket and residuum to firmly suspend the prosthesis; however, proper fit is a moving target, because residual limb volumes fluctuate by several percent over the course of each day. In an attempt to adapt to changing limb volumes, people with amputation use removable padding, called “socks”, to manually adjust the fit and fill of their socket. These adjustments are cumbersome: patients have to interrupt their activity, remove their prosthesis, add or remove socks, put their prosthesis back on, test the adjusted fit, and repeat until the interface feels comfortable (FIG. 1B). This adjustment process is enormously intrusive, and the lack of consistency is a major impediment to quality of life. Several technologies have emerged in recent years to address the challenges of limb volume change, including manually adjustable sockets, low-profile compressive sockets, and automated dynamic sockets. However, all of these technologies still rely on the soft tissues for suspension, and are therefore still prone to skin irritation, discomfort, pistoning, and pain (as in FIG. 1C).

In addition to the dangers surrounding pistoning, the majority of suspension systems also require patients to wear silicone liners, which bear their own clinical challenges. Liners are designed to stick to the skin and create a suction seal with the socket. To support this suction function, it is necessary that the liner material not “breathe”, which leads to overheating of the residuum and profuse perspiration. The hot, damp liner environment is uncomfortable and fosters ulceration and infection, which are of primary concern during prolonged socket use.

Socket issues are so profound that some patients are driven to pursue drastic surgically-invasive alternatives. Most notable among these is percutaneous osseointegration (OI), which involves a metal implant that is anchored to the residual bone and protrudes distally through the skin (FIG. 2); the prosthesis is then attached directly to the distal end of the implant. The obvious challenge associated with OI is the obligatory chronic perforation of the skin envelope, and the consequent infection risk (FIG. 2C). As a result, overall failure rate for OI is approximately 20%. Elevated healing requirements currently limit the use of OI in persons with dysvascular amputation, which is the most prevalent indication for limb loss. In addition, limitations in implant strength currently preclude all athletic activities: the Instructions for Use document for the only FDA-approved OI implant states, “the patient should never run, jump or climb, should always use a cane or crutches for longer walks, never lift or carry heavy items and never subject the [implant] to high torques.” These restricted activities are of crucial importance for patients looking to return to Service or other work, as well as for sustained cardiovascular health and quality of life.

Other examples of surgical interventions for prosthetic attachment include angulation osteotomy and T-prostheses, both of which create subcutaneous protuberances for mechanical anchoring of prosthetic sockets, and are typically used in the upper extremity. These approaches still require mechanical load transmission from the socket through a thinned layer of soft tissue, and are therefore susceptible to the same issues of discomfort and ulceration as conventional attachment systems.

Thus, there is the need in the art for prosthesis suspension systems that reduce skin irritation and/or ulceration, infection and activity restriction while not limiting the range of attachable prosthesis or attachment location. The present invention satisfies these needs.

### SUMMARY OF THE INVENTION

Aspects of the present invention relate to a magnetic prosthetic suspension system having a magnetic implant configured to be fixedly attached to bone at a target site of a subject, a socket configured to fit over the target site of the subject, and a magnet positioned on or within the socket, wherein the magnet is configured to generate a magnetic attractive force between the magnetic implant and the magnet that suspends the socket at the target site of the subject.

In some embodiments, the magnet is selected from the group consisting of an electromagnet, a permanent magnet, and an electro-permanent magnet. In some embodiments, the magnet comprises an array of permanent magnets. In some embodiments, the array of permanent magnets comprises one or more Halbach cylinders. In some embodiments, the array of permanent magnets comprises an inner cylinder of permanent magnets surrounded by an outer cylinder of permanent magnets, and wherein the array of permanent magnets is configured to produce an adjustable axial flux.

In some embodiments, the system has a motor configured to rotate at least one of the magnets. In some embodiments, the system has a motor configured to rotate the inner cylinder of permanent magnets relative to the outer cylinder of permanent magnets. In some embodiments, the system has a transmission configured to connect the motor to at least one of the magnets. In some embodiments, the system has a power source connected to the socket and configured to power the magnet. In some embodiments, the system has a computing device configured to instruct the transmission and motor. In some embodiments, the system has an array of sensors positioned in or on the socket comprising at least one of an accelerometer, a gyroscope, a load cell and a pressure sensor.

In some embodiments, the magnet is positioned at the distal end of the socket underneath the implant when the socket is positioned on the target site of the subject. In some embodiments, the system has a skin interface layer within the socket and configured to form an interface between the socket and the target site of the subject. In some embodiments, the socket comprises a receiver configured to engage a prosthetic attachment. In some embodiments, the magnetic implant comprises a biocompatible coating. In some embodiments, the magnetic implant comprises tissue in-growth features. In some embodiments, the magnet forms a cuff around the target site of the subject such that the cuff surrounds at least a portion of the magnetic implant when the socket is positioned on the target site of the subject.

Aspects of the present invention relate to a method for magnetic prosthetic suspension having the steps of attaching a magnetic implant to a bone at a target site of a subject, positioning a socket having a magnet over the target site, and generating a magnetic attractive force between the magnet and the magnetic implant, thereby suspending the socket from the target site.

In some embodiments, the ferromagnetic implant is implanted subcutaneously. In some embodiments, the method further has the step of modulating the magnetic force of the magnet during use. In some embodiments, the target site is a leg of the subject and the magnetic force is modulated to different values during one or more phases of gait of the subject.

In some embodiments, the method further has the step of positioning a skin interface layer between the socket and the limb. In some embodiments, the method further has the step of attaching a prosthetic attachment to the socket. In some embodiments, the modulated magnetic force produces an adjustable axial flux.

Aspects of the present invention relate to a permanent magnet assembly having a first plurality of permanent magnets forming an inner Halbach array cylinder configured to produce a predominantly axial flux, a second plurality of permanent magnets forming an outer Halbach array cylinder configured to produce a predominantly axial flux, wherein the inner Halbach array cylinder is nested within the outer Halbach array cylinder such that the inner Halbach array cylinder and the outer Halbach array cylinder are coaxially aligned and a relative rotation between the inner and outer Halbach array cylinders results in an adjustable axial flux.

Aspects of the present invention relate to a magnetic prosthetic suspension system including a magnetic implant configured to be fixedly attached to bone at a target site of a subject, a socket configured to fit over the target site of the subject, and any disclosed system or permanent magnet assembly on or within the socket, wherein the permanent magnet assembly is configured to generate a magnetic attractive force between the magnetic implant and the permanent magnet assembly that suspends the socket at the target site of the subject.

In some embodiments, the assembly further includes a first ring at least partially encasing the first plurality of permanent magnets, a second ring at least partially encasing the second plurality of permanent magnets, a bearing housing having a top surface in contact with a bottom surface of each of the first ring and the second ring, a bearing positioned at least partially within a bearing housing, and a bearing shaft having a top region and a bottom region, wherein the bearing shaft passes through the bearing and engages the first ring at the top region and is connected to the motor at the bottom region.

In some embodiments, the assembly has a motor connected to at least one of the inner Halbach array cylinder and outer Halbach array cylinder and configured to rotate the inner Halbach array cylinder and outer Halbach array cylinder relative to each other to produce the adjustable axial flux. In some embodiments, the assembly has a transmission. In some embodiments, the motor and transmission are configured to counterrotate the first and second rings at angular velocities that result in a net zero angular momentum. In some embodiments, the motor and transmission are non-backdrivable such that the torques generated by magnetic interactions between the first and second pluralities of permanent magnets do not cause rotation. In some embodiments, the bearing is positioned below the first and second rings. In some embodiments, the bearing is positioned at least partially within a gap between the first and second rings.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts a conventional suction suspension system (A), requiring a patient to add/remove socks (B) to maintain fit and prevent pistoning when their residuum changes volume. Poor socket fit can cause many problems, including skin irritation and ulceration (C).

FIG. 2 depicts a conventional osseointegration system (A), providing a direct mechanical connection between the bone and prosthesis (B), but requiring chronic perforation of the skin, creating a severe infection risk (C). Deep infection in the bone (C, top) is extremely dangerous, and can lead to implant loosening or removal, or even re-amputation.

FIG. 3 depicts a high-level illustration of an exemplary magnetic suspension system according to an aspect of the present invention (A). A prosthetic limb, such as a leg (B) or arm (C) is attached to a biological limb using magnetic attraction between a subcutaneous ferromagnetic implant and an external magnetic device.

FIG. 4 depicts the exemplary magnetic suspension system of FIG. 3A. The exemplary system uses an electromagnet in the socket to attract a ferromagnetic implant in the residuum. Because the implant is contained beneath the skin, this architecture provides robust attachment without infection risk. The exemplary system may have a socket design with external electromagnet hardware.

FIG. 5 depicts another exemplary suspension system.

FIG. 6A depicts exemplary implant geometries according to an aspect of the present invention. The depicted implant geometries include one ovular (FIG. 6B) in shape and the other circular (FIG. 6C) in shape. The hatched surfaces (bottom figures) are designed to mate with third-party intramedullary fixation hardware.

FIG. 7 depicts another exemplary implant with added features of holes to allow for surgical suturing to the implant and a porous coating to some surfaces of the implant to facilitate tissue ingrowth.

FIG. 8 depicts another exemplary implant composed of a ferromagnetic base and interchangeable top portions made from another material.

FIG. 9 depicts an exemplary external electromagnet assembly according to an aspect of the present invention. FIG. 9A shows the external electromagnet is an assembly of the electromagnet (core, coils, and shell), cooling structures, and attachment mechanisms. FIG. 9B illustrates the overall dimensions of the assembly. The molded insert is not included in the dimensions as it would be customized to fit each patient.

FIG. 10 depicts an exemplary solenoid implementation of an external electromagnet according to an aspect of the present invention.

FIG. 11 depicts the incorporation of exemplary secondary magnetic components into the residual limb.

FIG. 12 depicts an exemplary MagSwitch® working principle. When the top and bottom magnets are aligned (A), the device produces a maximum magnetic field. By rotating the top magnet 180 degrees relative to the bottom magnet (B), the individual magnetic fields destructively interfere with one another, and a minimum magnetic field is produced.

FIG. 13 displays an exemplary magnetic field of a linear Halbach array. The field above the array is greatly increased while little field is present below the array.

FIG. 14 depicts an exemplary axial flux field-adjustable Halbach cylinder according to an aspect of the present invention. The arrangement of the permanent magnet elements creates a maximum magnet field (A) when the inner and outer elements have aligned polarity, and a minimum field (B) when the rings are rotated relative to each other until the inner and outer elements have opposed polarities. The polarity of each element is shown by arrows (dot signifies out of the plane and cross signifies into the plane).

FIG. 15 shows an exemplary mounting structure or bearing assembly for a nested Halbach cylinder in the case of using a bearing external to the rings.

FIG. 16 shows an assembled exemplary mounting structure for the case of the bearing being below the rings.

FIG. 17 depicts another exemplary design for mounting the rings where the bearings are fit into a gap between the inner and outer rings.

FIG. 18 depicts an exemplary preliminary implant and electromagnet design according to an aspect of the present invention. Shown is the exemplary preliminary implant and electromagnet design (A) and finite element modeling (B) demonstrating that the system can feasibly produce the forces necessary for socket suspension (C).

FIG. 19 is an exemplary illustration of a preliminary dynamic (A) and a biomechanical model (B) of attractive force required to suspend sockets of different mass during the swing phase of gait (prosthesis mass was held constant).

FIG. 20 is an exemplary illustration of the force required to suspend a knee-ankle-foot prosthesis during walking. This uses the biomechanical model in FIG. 19 and data from a published gait dataset for persons with transfemoral amputation.

FIG. 21 is an exemplary illustration of the force required to suspend a knee-ankle-foot during the swing phase of walking for sockets of different mass, and the relationship between peak pulloff force during swing and socket mass.

FIG. 22 depicts an exemplary general electromagnet design composed of a core, copper coils, and a shell around the coils, along with the equivalent parameterized electromagnet geometry used for designing the system.

FIG. 23 is an exemplary parameter sweep depicting the effect of changing each parameter (described in FIG. 22) on the power required to produce the peak force during walking (from biomechanical analysis), the mass of the electromagnet, and the passive zero-current force produced by the permanent magnet core of this illustrative design.

FIG. 24 depicts a testbench validation setup. Shown is a testbench (A) validation of prototype implants with a custom electromagnet produced comparable results to an equivalent FEA model (B).

FIG. 25 displays the results for an optimized exemplary electromagnet. Shown are the force vs. current (A) and power vs. force (B) curves for the optimized electromagnet. The attractive force produced at zero current (pre-load force) is due to the use of a permanent magnet as the core material. Using a permanent magnet core also results in less power being required to reach low forces.

FIG. 26 displays the results for the power required to counteract the pulloff force of the socket over the whole gait cycle (stance and swing) for various subjects used in the biomechanical analysis. While peak power is around 250 W, power is only required for around a third of the gait cycle. This results in an inter-subject average power of around 32 W.

FIG. 27 displays the thermal analysis of the electromagnet simulated during continuous walking. With the use of a liner between the top of the electromagnet and the skin (already found in conventional sockets), the skin stayed below body temperature (37° C.) with both active or passive cooling.

FIG. 28 shows the placement of a femoral endoprosthesis (A), similar in size/shape to the disclosed invention, to successfully lengthen a short transfemoral residuum. In preliminary dissections, skin thickness (B) and potential implant geometries (C) are assessed.

FIG. 29 shows an exemplary surgical process for implant coverage investigated in a secondary dissection.

FIG. 30 depicts an exemplary, preliminary control framework according to an aspect of the present invention, and the results of finite element analysis modelling. A computational simulation of the dynamic control system was implemented (A) and has the potential to eliminate pistoning (B) in the presence of gait-relevant disturbance forces.

FIG. 31 shows the results for attractive force vs. rotation angle between the concentric discs of the adjustable axial-flux Halbach cylinder. In this configuration, the resultant magnetic field varies from maximum to minimum field strength over a 90 degree relative rotation between the cylinders.

FIG. 32 is a diagram of an example computing device in which aspects of the invention can be practiced.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

Disclosed herein is a novel system and method of suspending prosthetic limb devices from biological structures through magnetic attraction. In some embodiments, the system disclosed approach is unique and distinct as suspension forces are transmitted electromagnetically, rather than via direct mechanical contact. This fundamental difference makes these embodiments less likely to cause chronic skin irritation or ulceration. Conventional attachment systems and methods rely on transferring load through the soft tissues (skin, fat, muscle) to suspend the device from the body. The disclosed system transfers suspension loads directly to the skeletal system using magnetic attraction between a bone-anchored, subcutaneous ferromagnetic implant and an external magnetic device such as a permanent magnet or electromagnet. The disclosed novel suspension system and method has many advantages over conventional approaches by reducing soft tissue strain and the need to engage a large surface area of skin while more efficiently transferring load between the device and the body. It should also be appreciated that in some embodiments, suspension forces are transmitted electromagnetically and via direct or indirect mechanical contact. Accordingly, the present invention includes embodiments with suspension forces transmitted magnetically or electromagnetically only (i.e. without a direct or indirect mechanical contact), or in conjunction with a direct or indirect mechanical contact.

Conventional methods for attaching prosthetic limbs to the body involve loading the soft tissues (skin, muscle, fat) instead of directly connecting to bone. While bone is built to handle mechanical loading, interfacial soft tissues are rife with biological sensors that react to deformation, triggering pain and discomfort when loaded. In some cases, sustained loading of soft tissue can even lead to ulceration, which poses extreme health risks to patients. To alleviate soft tissue irritation, current attachment mechanisms are designed to distribute load over large surface areas, resulting in bulky, cumbersome systems that restrict natural movement and inhibit the skin's ability to sense the environment. The viscoelastic properties of soft tissues also add “slop” to the human-mechatronic system, reducing transmission efficiency by as much as 50% and creating unpredictable dynamics. In the case of prosthetic limbs, percutaneous osseointegration (OI), where direct skeletal attachment is achieved via a bone-anchored implant that protrudes through the skin, has been developed to eliminate soft tissue loading. OI comes with many disadvantages of its own, however, as it creates an infection-prone chronic wound (making it unsuitable in dysvascular amputation cases) and athletic activity is limited in users due to the risk of fracture at the bone-implant interface.

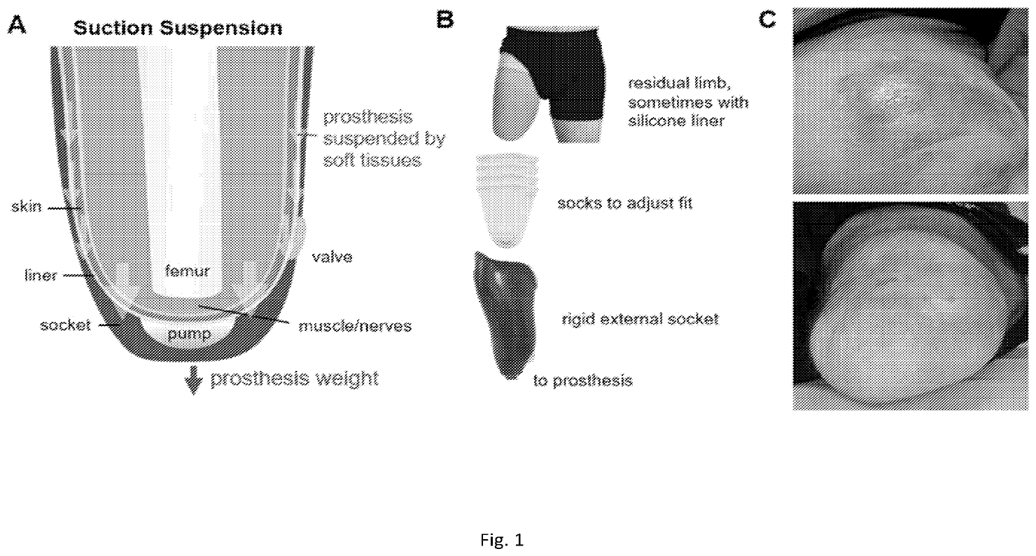

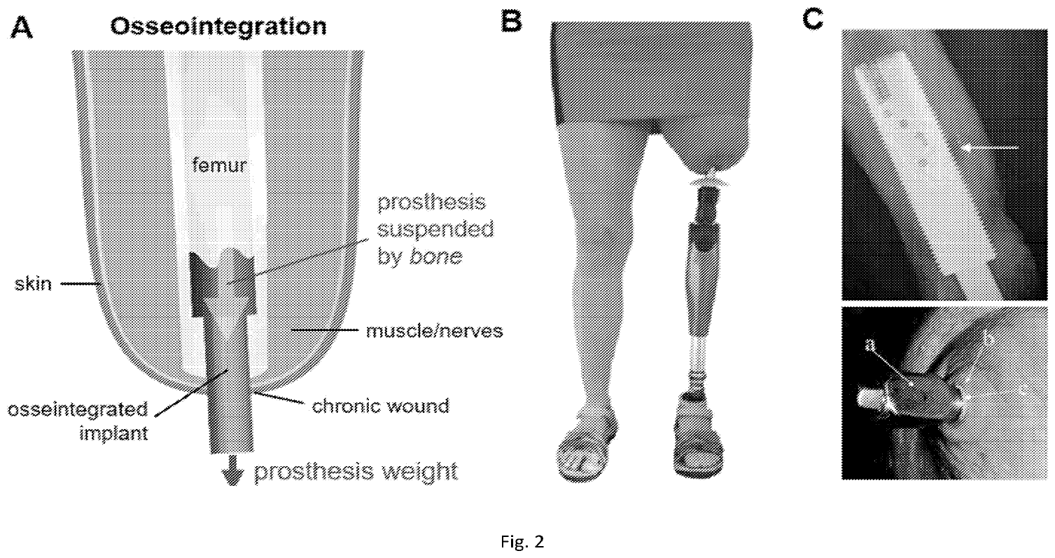

In some embodiments, the present invention relates in part to a new implant system that transmits magnetic forces to the skeletal system, and a novel controller capable of modulating the attractive force between an external electromagnet and a subcutaneous implant to maintain stable socket fit. Taken together, these two innovations represent a paradigm shift in the way rehabilitation devices attach to the body, toward a model that leverages parallel engineering of body and machine. Based on a thorough review of the academic literature, it is believed that ferromagnetic structures have never before been implanted into the body to facilitate electromagnetic attachment of prosthetic limb devices. Instead, previous attempts at magnetic socket suspension have focused on the interface between the socket and the liner, rather than the socket and the residual bone. While simplifying and improving the socket-liner interface is a worthwhile pursuit, such strategies still transmit load through the soft tissue via a conventional silicone liner; they are therefore subject to the same challenges of traditional suspension systems, including perspiration, pistoning, and loosening with volumetric changes (FIG. 1). The innovative use of electromagnetic attachment as contemplated herein allows for direct transmission of load to the endoskeleton, without requiring chronic skin perforation.

In some embodiments, the system does not generate repulsive forces. Repulsive forces have been the focus of previous work aimed at reducing compressive loads on the residuum.

Such “repulsive levitation” strategies face significant barriers to translation, due in large part to their reliance on a high-strength permanent magnet implanted in the residual bone. An implanted permanent magnet strong enough to suspend a person in their socket would also be strong enough to cause significant tissue damage in the presence of external ferromagnetic material (e.g. the refrigerator door) by generating several hundred pounds of attractive force. In contrast, embodiments of the system presented herein tackles the problem of socket fit from a suspension perspective, and therefore does not require implantation of a permanent magnet. Instead, the present invention uses an electromagnet to induce temporary magnetism in a passive ferromagnetic material, such as ferritic stainless steel (SS) with a biocompatible coating. However, it should be appreciated that in some embodiments, the system may alternatively utilize an implanted permanent magnet. In some embodiments, the system may also alternatively generate repulsive forces.

In some embodiments the disclosed system, such as an electromagnetic suspension system, offers significant benefits over state-of-the-art suspension. First, the approach has the potential to drastically improve fit and eliminate pistoning, by maintaining a slight compressive “preload” in the socket during the swing phase of gait. This is especially beneficial in cases of short or conical residual limbs, where suspension is a dominant challenge. In addition, the present suspension method is agnostic to changes in residual limb volume, which should stabilize fit throughout the day and enable patients to use their sockets for longer periods without adding or removing temporary padding. The present methodology also obviates the suction function of the traditional liner, paving the way for lower-profile sockets and porous liners that pad the residuum without creating an ulceration hotbed.

Although the primary purpose of the novel implant is to support suspension of the prosthesis during the swing phase of gait, the disclosed approach also has significant implications for the stance phase. For instance, by eliminating pistoning, the system avoids dangerous shock loads during heel strike, which will substantially reduce peak stance-phase stresses on the residuum. In addition, the implant's distal end will have a large surface area, to help distribute compressive loads and avoid stress concentrations in the tissue. The disclosed design also explores oblique implant geometries that prevent rotation of the prosthetic socket with respect to the residual femur, to stabilize the prosthesis in the context of twisting motions. In some embodiments, the electromagnet may be powered off (or operating at very low current) during the stance phase, so as not to create additional stress on the residuum.

Unlike OI, the present invention is not subject to the pernicious transcutaneous infection issue, which seems inescapable in the near term and may perpetually limit the applicability of OI in the dysvascular population. In some embodiments, the use of electromagnetic attraction as the anchoring force provides the key socket-suspension benefits of OI without perforating the skin envelope. In this way, the system circumvents the dominant issues that are inherent to OI, eliminating the transcutaneous infection pathway and associated risk of implant failure. Further, it is contemplated herein that bony suspension may increase patient tolerance of prosthetic mass, as supported by evidence showing that OI, which also leverages suspension from bone, increases satisfaction, embodiment, and use of the same prosthetic devices that are described as “too heavy” when used with conventional sockets.

### Magnetic Suspension System

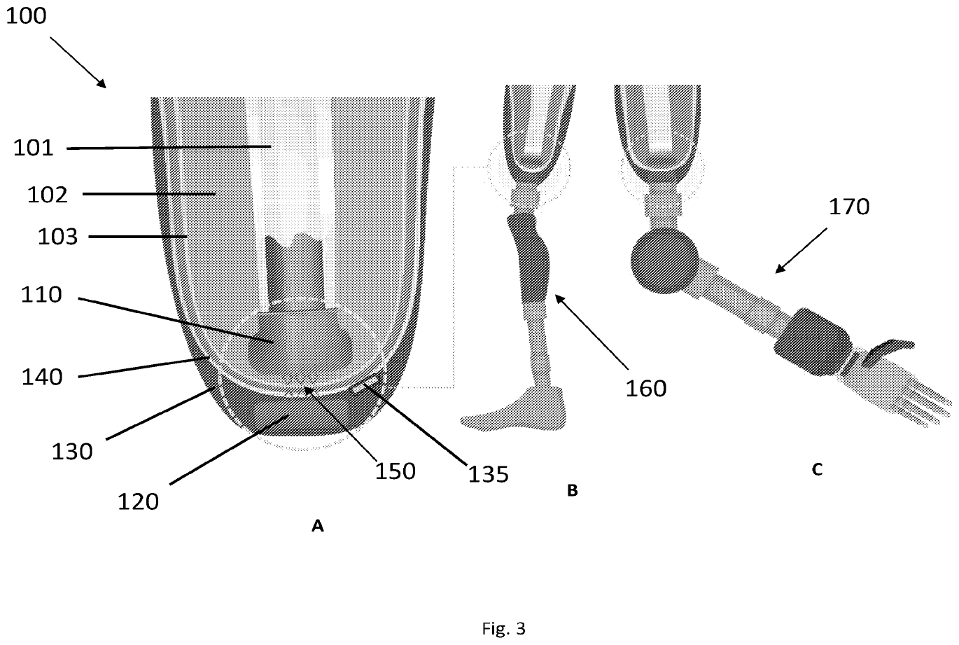

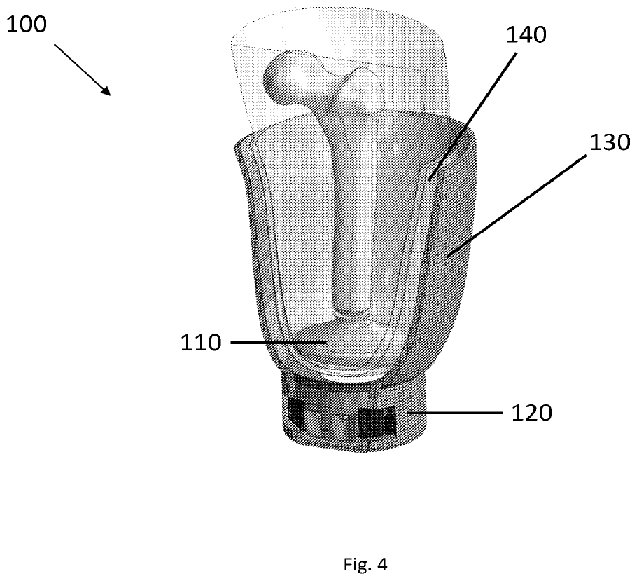

In light of these challenges, disclosed is a novel socket suspension system and method that enables direct transmission of load to residual bone, while maintaining a sealed skin envelope. In some embodiments, the disclosed approach leverages an electromagnet in the distal socket to create an attractive force between the socket and a ferromagnetic implant in the residual bone. In some embodiments, the system comprises a subcutaneous ferromagnetic implant, anchored to the amputated bone, and an external electromagnet embedded in the distal socket. In some embodiments, the implant may comprise a ferritic stainless steel with a biocompatible coating, and may be designed to be placed in the subject either at the time of primary amputation, or in a dedicated procedure. In some embodiments, the external electromagnet is positioned such that the attractive force generated between it and the implant acts along the femur's primary load-bearing axis (FIGS. 3 and 4). The magnitude of this attractive force may be controlled by modulating the electrical current through the electromagnet's coils, based on feedback from one or more sensors within the socket. The controller may be designed to maintain a slight preload on the residual limb, to ensure continuous prosthetic suspension. Importantly, in embodiments where the system is built around an electromagnet, the attractive force may easily be “turned off” by disconnecting the current source.

In some embodiments, the magnetic suspension system comprises a subcutaneous ferromagnetic implant and an external magnetic device that attach a prosthetic limb to a biological limb using a magnetic attraction between the implant and the magnet. In some embodiments, a target site on a subject is chosen that may be near a site of amputation, near a bone at a site of an amputation and/or relating to a region, location or site of interest on a subject for prosthetic attachment. For example, relating to a site of amputation involving a limb, residual limb, joint, appendage, knuckle and/or digit. As contemplated herein, the magnetic suspension system may be used in various prosthetic applications on any limb and at any amputation level, including but not limited to: through hip, above knee, through knee, below knee, through ankle, mid foot, through shoulder, above elbow, through elbow, below elbow, full or partial hand, digital (fingers/toes).

Referring now to FIG. 3A, a magnetic suspension system 100 may generally include a magnetic implant 110 configured to fixedly engage and attach to the distal end of a subject's bone 101, such that implant 110 resides within the soft tissues 102 of the limb beneath the skin 103. The system 100 further includes one or more magnets 120 embedded in, attached to or on, or otherwise associated with a prosthetic socket 130. Alternatively, magnet 120 may be embedded in, attached to or otherwise associated with a socket liner 140. In some embodiments, magnets 120 are positioned on or within both socket 130 and liner 140. A magnetic field 150 generates the desired attractive force between implant 110 and magnet 120. This attractive force holds the subject's stump or residuum into place within socket 130 associated with a prosthetic leg 160 (FIG. 3B) or arm 170 (FIG. 3C), for example. System 100 may additionally include one or more sensors 135 which may be positioned on or within socket 130 and/or liner 140. Exemplary sensors 135 may include, without limitation, load cells, pressure sensors, ultrasonic sensors, hall effect sensors, electromyography sensors, gyroscopes, accelerometers, and magnetometers.

Eliminating soft tissue suspension with the magnetic attachment system disclosed herein allows for more freedom in socket design. For example, because the socket and/or liner does not need to cover large amounts of the residual limb surface or maintain a seal for suction when using embodiments of the magnetic attachment system, cutouts for sensors or ventilation may be incorporated into the socket. These cutouts may leave various areas of skin exposed for improved sensorization of residual limbs. In some embodiments, sensors 135 may be placed directly on the skin without needing to be underneath the liner or the socket as these structures can cause motion artifacts and noise in sensor signals. In some embodiments, these sensors may further be used within the control system for the attachment system or the attached prosthetic limb. These sensors may also be used to measure the quality of the attachment to warn the user of suboptimal attachment or disconnection.

Accordingly, aspects of the present invention relate to a socket of the magnetic suspension system. In some embodiments, socket 130 is placed over a residual limb such that socket 130 forms a volume to support a mechanical interface between a residual limb and prosthetic attachment. In some embodiments, socket 130 includes or is associated with liner 140 that is in contact with the skin. In some embodiments, socket 130 is a molded insert that is sized appropriately for the residual limb, such that the socket 130 is uniformly supported during normal gait or movement activity. In some embodiments, socket 130 is generally cylindrical in shape with a semi-circular cavity to surround the residual limb of the subject. In some embodiments, socket 130 is composed of materials such as, but not limited to, plastics, metals, composites, foams and any combination thereof. In some embodiments, socket 130 includes automated features to provide dynamic support to the limb of the subject in response to change in residual limb volume.

In some embodiments, socket 130 may incorporate active elements (e.g. motors, pumps) to adjust fit. Such active elements may be controlled manually by the subject or automatically by the computerized control system as described herein. In some embodiments, non-powered components (e.g. cables, pumps) may also be used to adjust fit. These adjustments may act upon moveable panels, an actuated sidewall, or a clamshell mechanism to better secure around the limb. Socket 130 geometry may further be made less intrusive on the pelvic area in the case that the patient can end bear on the residual limb because the ischium would not be needed to support weight bearing. In some embodiments, liners 140 may be eliminated from socket 130 or included for cushioning purposes. In some embodiments, liner 140 materials may be breathable, porous, and/or contain cutouts to increase ventilation and reduce sweating. Active cooling, thermoelectric coolers, or airflow channels may be incorporated into socket 130 to better cool the residual limb.

As shown in FIG. 4, system 100 may be integrated into socket 130 with magnet 120 positioned externally to socket 130. In some embodiments, socket 130 does not include any cutouts or additional features that may optionally be included.

In some embodiments, liner 140 forms an interface between socket 130 of magnetic suspension system 100 and the skin of the subject. In some embodiments, liner 140 is modifiable in shape, thickness and/or material to appropriately fit the subject. In some embodiments, liner 140 comprises a biocompatible material suitable for prolonged use in direct contact with skin. In some embodiments, liner 140 is dynamic. In some embodiments, liner 140 is user-adjustable. In some embodiments, liner 140 comprises automated features to provide dynamic support to the limb of the subject in response to change in residual limb volume. In some embodiments, liner 140 is eliminated from socket 130 and/or incorporated into socket 130 as an integrated component.

In some embodiments, the magnetic suspension system also comprises a power source, a computing device, a motor, a transmission, and a pyramid receiver for affixing various prostheses. As contemplated herein, system 100 may be powered by the same batteries used in powered prostheses, and can share such a battery with the prosthetic leg. Alternatively, a dedicated battery may be housed in or on socket 130, attached to the pylon that connects socket 130 to the prosthesis, or worn separately on the waist. This implementation can be used in any limb and at any amputation level, including, but not limited to: through hip, above knee, through knee, below knee, through ankle, mid foot, through shoulder, above elbow, through elbow, below elbow, full or partial hand, digital (fingers/toes).

As mentioned previously and referring again to FIG. 3A, system 100 includes magnetic implant 110 which attaches to the distal end of a subject's bone, such that implant 110 resides within the soft tissues of the limb or residuum beneath the skin. In some embodiments, implant 110 comprises a magnetic material. In some embodiments, implant 110 comprises a soft magnetic material. In some embodiments, implant 110 comprises a hard magnetic material. In some embodiments, implant 110 comprises a magnet. In some embodiments, implant 110 comprises one or more ferromagnetic materials. In some embodiments, implant 110 comprises one or more paramagnetic materials. In some embodiments, implant 110 comprises one or more diamagnetic materials. In some embodiments, implant 110 comprises one or more non-ferromagnetic materials and one or more ferromagnetic materials. It should be appreciated that magnetic implant 110 may comprise any magnetic material, provided the magnetic material is either a biocompatible material, or the implant includes a biocompatible coating or shell material encapsulating the magnetic material. For example and without limitation, such materials may include rare-earth magnets (e.g. NdFeB, SmCo), ferritic steel, ferritic stainless steel, Hiperco® magnetic alloys, permalloy, mu-metal, or supermalloy. In certain embodiments, implant 110 is composed of 400 series stainless steel. In certain embodiments, implant 110 is biocompatible through the use of a fully biocompatible material or with biocompatible coatings such as TiN. Sections of implant 110 may be porous and/or coated with a material configured to facilitate tissue ingrowth. Permanent magnet materials may also be used within portions of implant 110 to assist in orientation of an external device onto the limb.

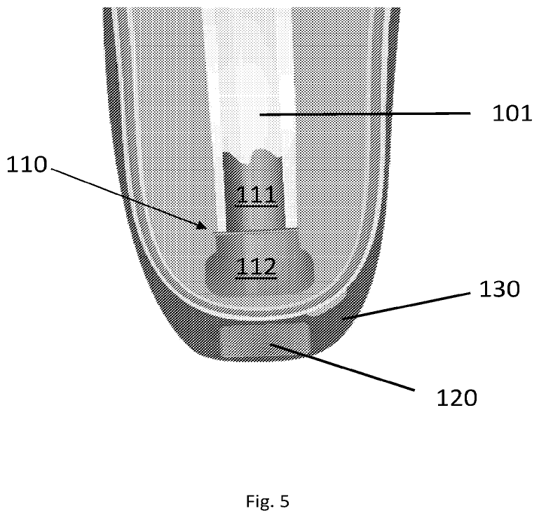

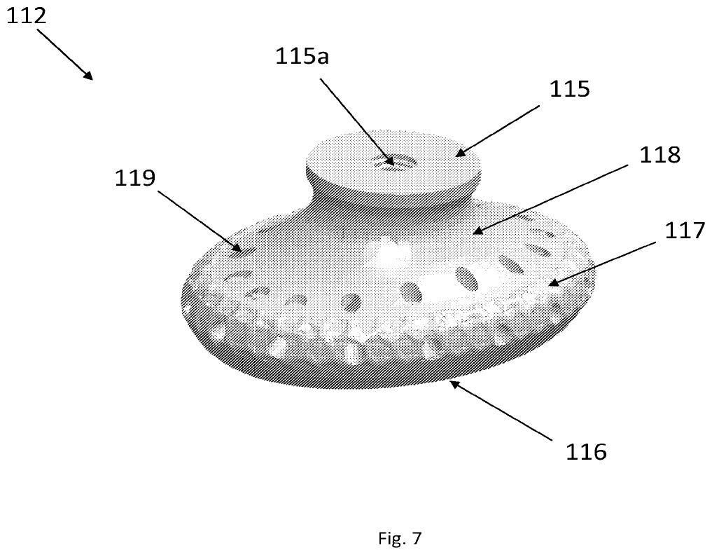

As shown in FIG. 5, implant 110 may include a body with a proximal or first end fixation component 111 which may function as a stem and may be configured for intramedullary fixation (into bone 101), and a distal or second end magnetic component 112 for providing a volume of magnetic material used for magnetic attractive force when a magnetic field is generated between implant 110 and magnet 120. In some embodiments, proximal and distal end components 111 and 112 form a single unit. In some embodiments, proximal and distal end components 111 and 112 are separate and engageable components. For example, proximal end fixation component 111 may include a threaded bolt/hole at its distal end that can engage a complementary threaded bolt/hole (e.g. 115a of FIG. 7) at the proximal end of distal end magnetic component 112. In this manner, optional sized or material make magnetic components 112 can be selected and utilized to pair with optional sized fixation components 111 at the time of implantation according to the need of the subject.

In some embodiments, fixation component 111 comprises various shapes, sizes and profiles for affixation to intramedullary bone locations. For example, fixation component 111 to be inserted in the distal end of the femur may comprise a tapered design that has one wider end and one narrower end.

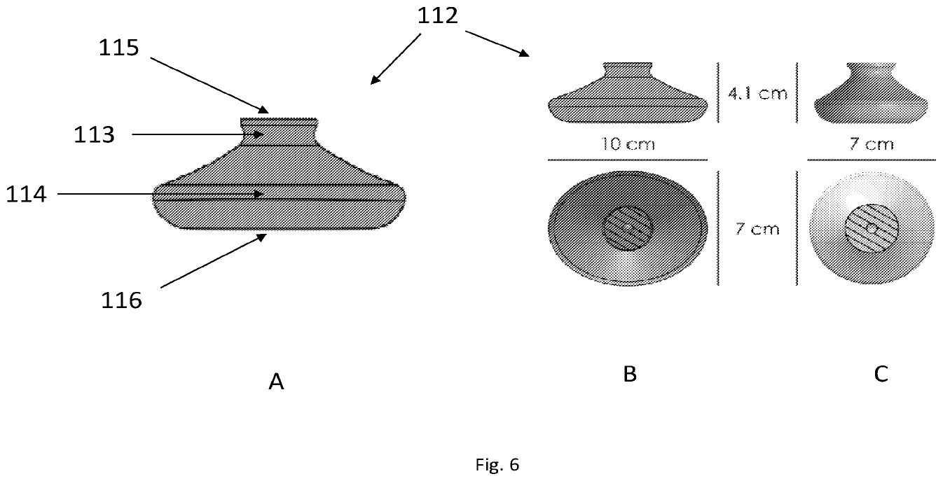

Referring now to FIGS. 6A and 6B, distal end magnetic component 112 of implant 110 includes a proximal region 113 and a distal region 114, and further includes a proximal surface 115 and a distal surface 116. As explained above, proximal surface 115 may include any engageable mechanism 115a to engage with proximal end fixation component 111. Such engageable mechanisms may be a permanent engagement or a releasable engagement. Exemplary engagement mechanisms may include, without limitation, adhesives, threaded bolt/screw, pins, welding, press fits, and the like.

It should be appreciated that distal surface 116 has the greatest effect on the strength of magnetic attraction. In some embodiments, distal surface 116 may be as large a surface area as possible while still allowing for skin closure around implant 110. A large distal surface 116 also increases the potential that the patient or subject could end bear using system 100. End bearing, where the body weight is supported by the distal end of the residual limb, decreases the load at the socket 130 brim in the groin and ischial area. This can allow for less intrusive and more comfortable socket 130 architectures. The overall shape of distal end magnetic component 112 of implant 110 can also be used to provide rotational stability of socket 130 if implant 110 is not axially symmetric. This may be achieved by an ovular magnetic component 112 cross-section (FIG. 6B) instead of a circular one (FIG. 6C). In this case, rotation of socket 130 would be inhibited by the compression of soft tissue between the sides of implant 110 and socket 130 as they become misaligned. In various embodiments, the cross-sectional shape of magnetic component 112 may be circular, ovular, rectangular, irregular, or any other desired shape. The general shape of magnetic component 112 may be bulbous. It should further be appreciated that distal region 114 of magnetic component 112 may be larger than proximal region 113, so as to create an increased area of distal surface 116. In some embodiments, the average diameter of distal surface 116 may be between 0.25 and 25 cm. It should be appreciated that the average diameter may be sized according to the anatomy of the implantation site. In some embodiments, the diameter of distal surface 116 is equal to or about equal to the diameter of magnet 120. In some embodiments, distal surface 116 is smooth. In some embodiments, distal surface 116 may include a textured surface (e.g. indentations, protrusions, ridges, grooves, etc.). In some embodiments, distal surface 116 is flat. In some embodiments, distal surface 116 may be curved, such as being concave or convex. In some embodiments, distal surface 116 may be flat in its central region and curved about its periphery. In some embodiments, distal surface 116 may be perpendicular to a longitudinal axis through implant 110. In some embodiments, distal surface 116 is not perpendicular to the longitudinal axis through implant 110.

In some embodiments, the volume of magnetic component 112 may be axially asymmetric or contain features that allow for rotational constraint within socket 130. In some embodiments, the volume of magnetic component 112 may resemble anatomical structures such as the femoral condyles. In some embodiments, the volume of magnetic component 112 may comprise a contoured region to improve implant integration and increase comfort for the subject. Additionally, implant 110 length may be sized when possible (e.g. amputation due to sarcoma) such that the residual limb is a preferred length. In some embodiments, the volume of magnetic component 112 is ovular in shape. In some embodiments, the volume of magnetic component 112 is circular in shape. In the embodiment shown in FIG. 6B, the ovular shaped volume may be about 4 cm high, by 10 cm wide, with a depth of about 7 cm. In the embodiment of FIG. 6C, the circular shaped volume is about 4 cm high, by 7 cm wide, with a depth of about 7 cm. In some embodiments, the volume of magnetic component 112 may have a cross section up to 20 cm across in either direction. In some embodiments, magnetic component 112 may be custom sized to each patient based on the patient's anatomical measurements. These measurements may include thigh diameter/width, width between femoral condyles, etc.

In some embodiments and as shown in FIG. 7, magnetic component 112 of implant 110 comprises a textured surface, such as embossed or debossed features to aid in implantation, osseointegration and/or bone ingrowth. In some embodiments, magnetic component 112 comprises a partially non-textured surface region 118, and a partially textured surface region 117. In some embodiments, magnetic component 112 comprises a roughened titanium surface and/or coating. In some embodiments, magnetic component 112 comprises a porous surface. In some embodiments, the implant comprises a metal-bead surface. In some embodiments, magnetic component 112 comprises a grit-blasted surface. In some embodiments, magnetic component 112 comprises a textured surface region that may be created by the means of sinter coating, chemical etching, abrasive roughening and/or other methods. In some embodiments, magnetic component 112 comprises bone-ingrowth features to aid in the permanent fixture of implant 110 to bone. In some embodiments, a portion of magnetic component 112 comprises a honeycomb structure to promote bone ingrowth. In some embodiments, magnetic component 112 may include one or more suture holes or posts 119 for looping a suture around or through.

As mentioned elsewhere wherein, in some embodiments implant 110 comprises a distal component 112 or volume of magnetic material that mates with intramedullary fixation component 111 or other hardware. In some embodiments, implant 110 comprises features for forming a rigid connection with intramedullary fixation hardware. For example, implant 110 may comprise a stem that is implanted into an intramedullary bone location using an installation tool and/or hammer, thereby creating a rigid post to permanently affix a magnetic volume. In some embodiments, implant 110 comprises features for affixing an installation tool, such as, but not limited to, threads, grooves, lumens, channels, embossed and/or debossed features. In some embodiments, the features for affixing an installation tool are used after the installation to affix the magnetic volume. In some embodiments, the stem and volume of magnetic material are permanently affixed with one or more threaded connections. In some embodiments, the stem and volume of magnetic material are permanently affixed with tongue-and-groove features and/or threads.

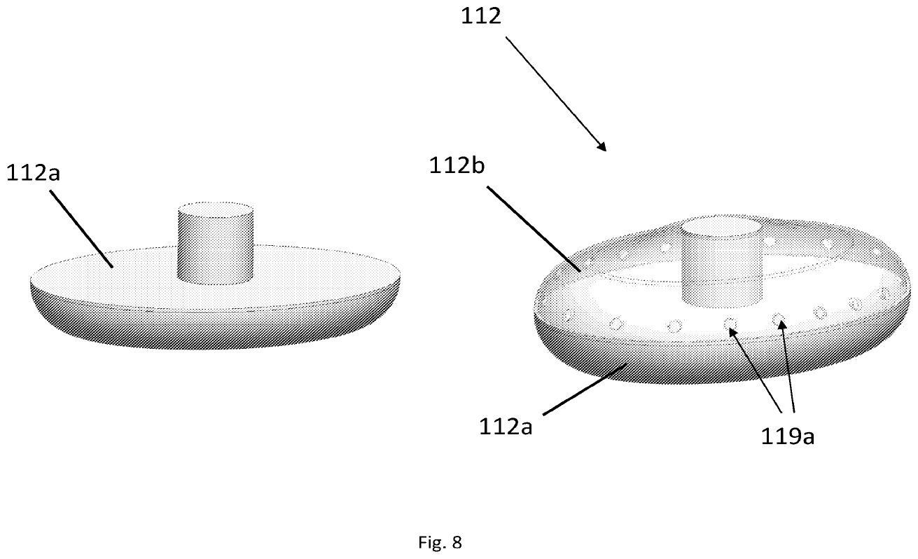

In some embodiments and as shown in FIG. 8, magnetic component 112 of implant 110 comprises a volume of magnetic material 112a along with a volume of non-magnetic material 112b. In this case, magnetic volume 112a is used to generate the magnetic attraction force with an external magnet 120, and the non-magnetic volume 112b is used to form implant 110 into a shape suitable for limiting the risk of implant extrusion. This non-magnetic cover 112b may also be made of a low-density material to reduce the overall mass of implant 110, such that the portions that need to be magnetic are minimized. In some embodiments, magnetic and/or non-magnetic volumes 112a and 112b may contain features such as porous coatings, or features for affixing soft tissues such as suture holes 119a.

As described herein, aspects of the present invention relate to magnets and one or more magnetic fields generated by one or more magnets. In some embodiments, magnet 120 associated with socket 130 may be a permanent magnet, an electromagnet, or a combination of permanent magnets and electromagnets to generate one or more magnetic fields. In some embodiments, magnet 120 may comprise an array of magnets. Magnet 120 of system 100 may comprise any combination of individual permanent magnets, individual electromagnets, and magnet arrays. In some embodiments, magnet 120 is a permanent magnet and produces a significantly lateral magnetic field around the target site. In some embodiments, magnet 120 is an electromagnetic and produces a significantly vertical magnetic field around the target site. In some embodiments, magnet 120 is one or more electromagnets for providing a dynamic lateral and vertical force to implant 110.

In some embodiments, magnet 120 comprises a ferromagnetic shell around an external electromagnet to direct the magnetic field towards implant 110 while reducing the field in all other directions. This creates an effectively one-sided magnetic field to reduce interference with components placed laterally or below the electromagnet. In some embodiments, magnet 120 comprises an electromagnet with a permanent magnet core. In this event, the magnetic field strength due to the core provides a specific preload force on implant 110. In some embodiments, the magnetic core of magnet 120 is sized such that the preload is of similar magnitude to the suspension force required to support the prosthetic limb at rest.

In some embodiments, the core of magnet 120 may comprise magnetic, non-magnetic, or a magnetized material (e.g. NdFeB rare-earth magnet). Depending on the core material, the core may produce its own magnetic field and/or influence the magnitude and direction of the magnetic field produced by the coils of the electromagnet. For example and without limitation, the core may be made up of rare-earth magnets (e.g. NdFeB, SmCo), ferritic steel, ferritic stainless steel, Hiperco® magnetic alloys, permalloy, mu-metal, or supermalloy. The core shape and material may be used to direct and focus the magnetic field of the electromagnet as well. Non-limiting examples of core cross-sectional shape could be rectangular, circular, or ovular, and could incorporate features protruding upwards from the top of the magnet 120 such as conical or pyramidal sections to further focus the field.

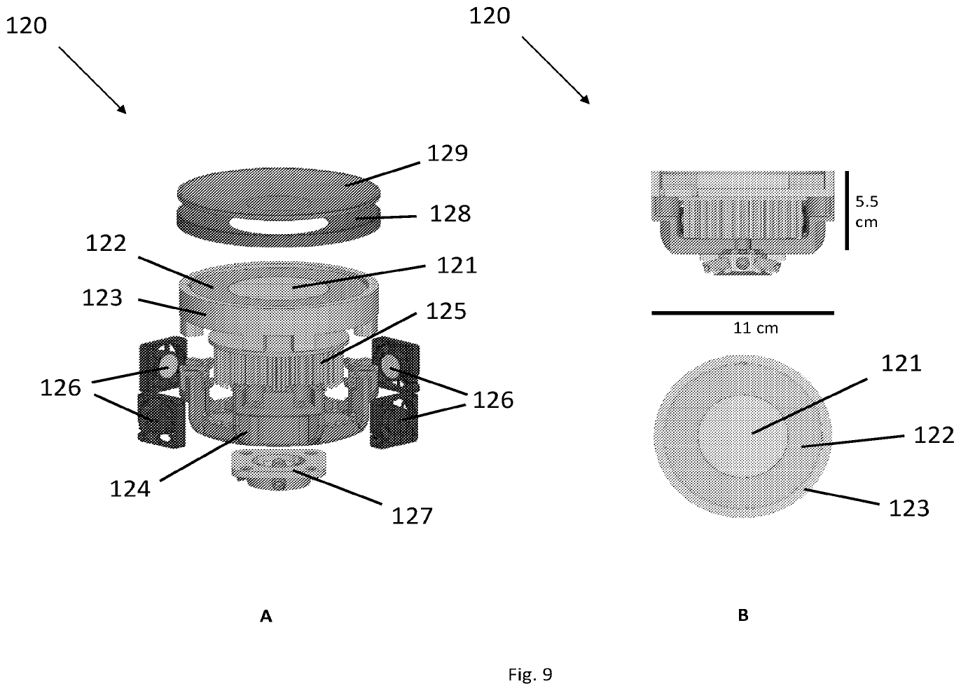

Given the unique requirements of this application, magnet 120 may include a novel and purpose-built electromagnet that minimizes the mass and the power required to suspend a prosthesis. As shown in FIG. 9, magnet 120 may include a core 121, coils 122, and a shell 123, which may sit between a molded insert 128 and liner 129, and a cooling structure or housing 124. Coils 122 may be wound around core 121 to modulate the net magnetic field depending on the direction and magnitude of current applied to coils 122. In some embodiments, core 121 is a permanent magnet. In some embodiments, core 121 is an electromagnet. In some embodiments, core 121 is made of a magnetic and/or non-magnetic material. In some embodiments, coils 122 are electromagnet coils. In some embodiments, coils 122 are a permanent magnet. In some embodiments, core 121 is made of a non-magnetic material, and as such the field generated by coils 122 will be the only field. In embodiments where a ferromagnetic material is used for core 121, then core 121 will amplify and concentrate the field produced by coils 122. In embodiments where core 121 is made of a magnetized material, one direction of current flow through coils 122 will produce a magnetic field that will enhance the core's own field, while in the opposite direction the current will produce a magnetic field that will counteract the core's own field. Coils 122 and core 121 are generally contained within shell 123 for purposes of structure. In embodiments where shell 123 is a ferromagnetic material, it will concentrate the magnetic field above magnet 120 and shield the environment from the produced magnetic field in all other directions. Due to the heat dissipation in coils 122, housing 124 is connected to the bottom of shell 123 and may further contain a heat sink 125 and one or more fans 126 to provide adequate cooling. A pyramid receiver 127 may be used to attach the prosthetic device to magnet 120. Molded insert 128 is fit to the patient's residual limb and could provide a space for sensors. In some embodiments, molded insert 128 is shaped such that it forms the bottom of the socket that would be in contact with the residual limb.

In some embodiments, core 121 may be made of any magnetic material, including non-ferritic, ferritic, or a permanently magnetic material. In some embodiments, coils 122 are conductive wires wound around core 121. In some embodiments, shell 123 is configured as a holder or frame for coils 122 and core 121. Shell 123 may be made of any magnetic or non-magnetic material. In some embodiments, shell 123 is made of a ferromagnetic material, such as ferritic stainless steel.

In some embodiments and as shown in FIG. 9B, magnet 120 may be sized between 0.25 and 25 cm in average diameter and 0.25 and 10 cm in height. In some embodiments, magnet 120 has an average diameter that is equal or about equal to the average diameter of distal surface 116 of implant 110. In some embodiments, magnet 120 is about 11 cm wide and 5.5 cm tall. The actual dimensions of magnet 120 will further be dependent on body part application and the size of the subject.

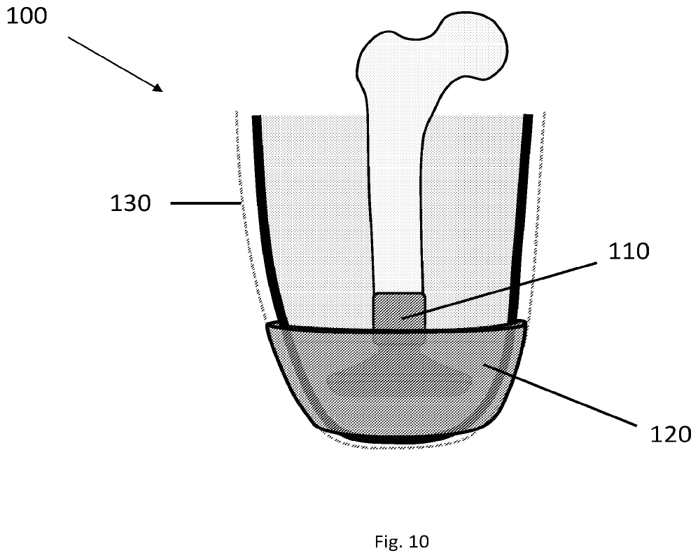

In some embodiments and as shown in FIG. 10, magnet 120 may be a solenoid cuff that wraps around the side of the residual limb for prosthetic attachment, rather than being housed below the limb. This takes advantage of the magnetic field inside a solenoid being near constant over the axial length of the solenoid, such that the attractive force on implant 110 does not depend on the gap distance between the bottom of implant 110 and the skin. In such embodiments, magnet 120 includes coils of an electromagnet that are wound around the outside of the residual limb to form the solenoid arrangement, with implant 110 taking the role of the solenoid plunger and the cuff forming the basis for an armature such that the attractive forces pulls implant 110 further into the solenoid cuff.

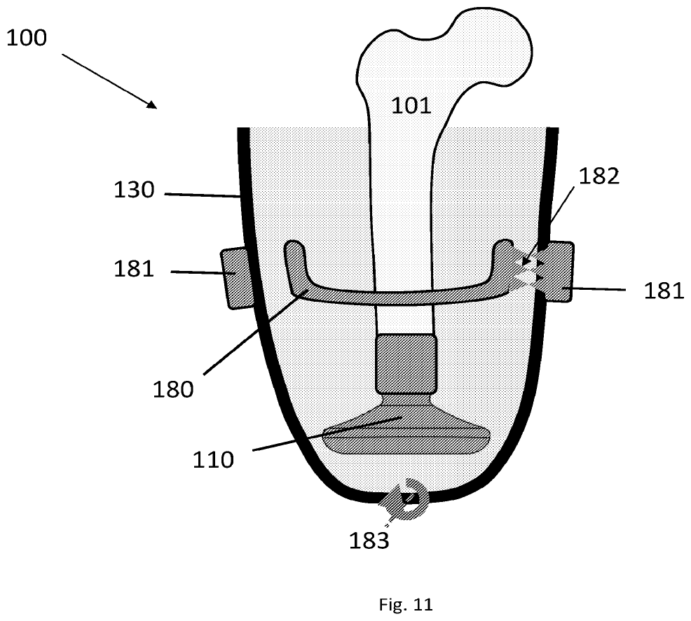

In addition to the primary magnetic components that would handle the suspension loads of the prosthetic limb, secondary magnetic components may be incorporated into the limb to improve stability of the attachment components. As illustrated in FIG. 11, secondary components such as secondary implant 180 located throughout the limb can create additional areas of load transfer via magnetic interaction 182. External magnets 181 within or on socket 130 may create magnetic interaction 182 with secondary implant 180 to provide horizontal or rotational stabilization. This would be useful for transferring torque 183 or torsion loads from the prosthetic device to the patient's skeletal system.

This implementation, as well as any other implementation described herein for magnet 120, can be used in any limb and at any amputation level, including but not limited to: through hip, above knee, through knee, below knee, through ankle, mid foot, through shoulder, above elbow, through elbow, below elbow, full or partial hand, digital (fingers/toes).

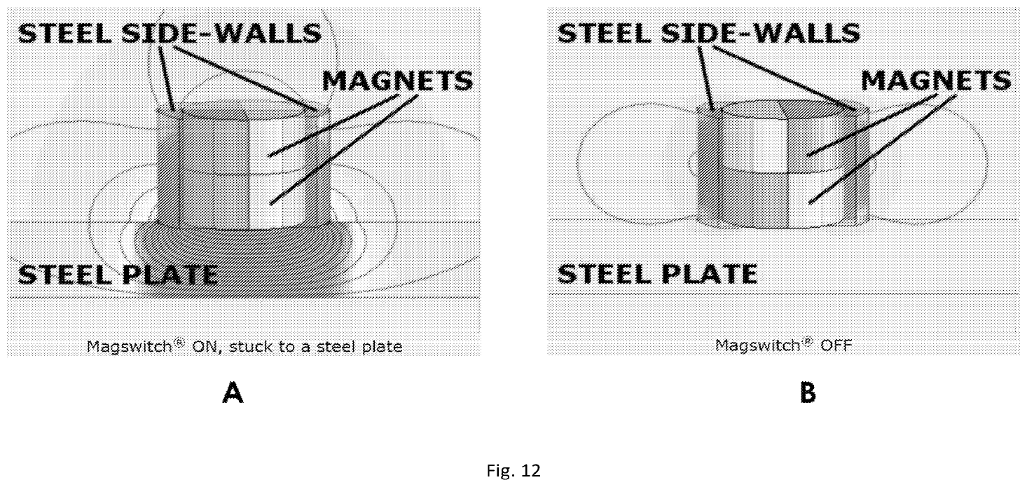

In some embodiments, instead of modulating a magnetic field by varying the amount of current flowing through loops of wire, the strength of a magnetic field can also be controlled by changing the relative orientations of multiple permanent magnets within an array. This is the working principle behind the MagSwitch® device, wherein the magnetic field can be switched on and off depending on the alignment of the north and south poles of two stacked magnets (FIG. 12). When the two magnets have aligned poles (FIG. 12A), the magnetic fields of each magnet add together to produce a net magnetic field of high strength. Rotating the top magnet by a half turn (which may be achieved by a mechanical crank) causes the poles to oppose one another, and the net field produced has a low strength (FIG. 12B).

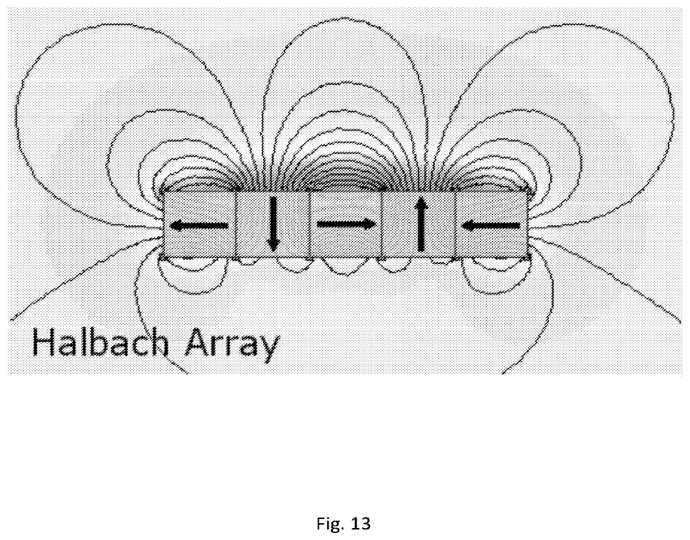

In cases requiring high field strengths, a magnet array configuration called a Halbach array is used (FIG. 13). The Halbach configuration is composed of adjacent permanent magnets with rotated polarities, which together create a net magnetic field that is one-sided: a strong field is created above the array and a weak field is present below the array. The field above the array has the additional characteristic of extending farther out from the surface, making this configuration ideal for magnetic attraction over a gap.

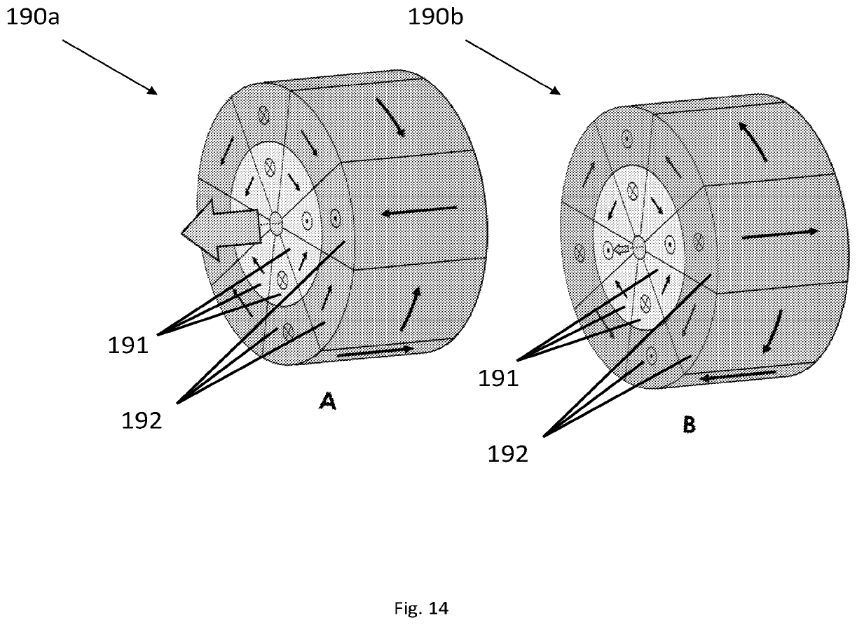

Accordingly, in some embodiments system 100 includes a novel design and application of field-adjustable permanent magnet arrays, including field-adjustable Halbach cylinders to generate axial magnetic fields within assistive devices. As shown in FIG. 14, are two concentric Halbach cylinders 191 and 192 shown in positions 190a and 190b. For example, included is a first internal set of magnets 191 forming an inner cylinder and a second outer set of magnets 192 forming a cylinder and surrounding inner cylinder 191, where first set 191 of magnets and second set 192 of magnets are coaxially aligned. The permanent magnet elements in each cylinder 191 and 192 are oriented such that the resultant magnetic field is oriented axially along the cylinder (arrows). The design of FIG. 14 takes advantage of the Halbach configuration as a strong field would be created above the cylinder that extends farther than a single magnet of the same size, while the field below is weak and will have little interaction with the environment. By rotating inner cylinder magnets 191 and outer cylinder magnets 192 such that the inner and outer magnets are aligned (190a) or opposed (190b), the field strength above the magnet is varied between a maximum (190a) and a minimum (190b), respectively. The specific values of these field maxima and minima are determined by the geometries, magnet grades, and relative sizing of the inner and outer rings 191 and 192. The rotational excursion required to go from a maximum to a minimum field is determined by the number of magnetic segments in each ring. The implementation shown in FIG. 14 has eight segments per ring, which results in a 90°angular rotation required to adjust the field from a maximum to a minimum. In some embodiments and without limitation, each ring or cylinder may have 4, 8, 12, 16, 20, 24, 28, or 32 magnet segments of alternating polarity.

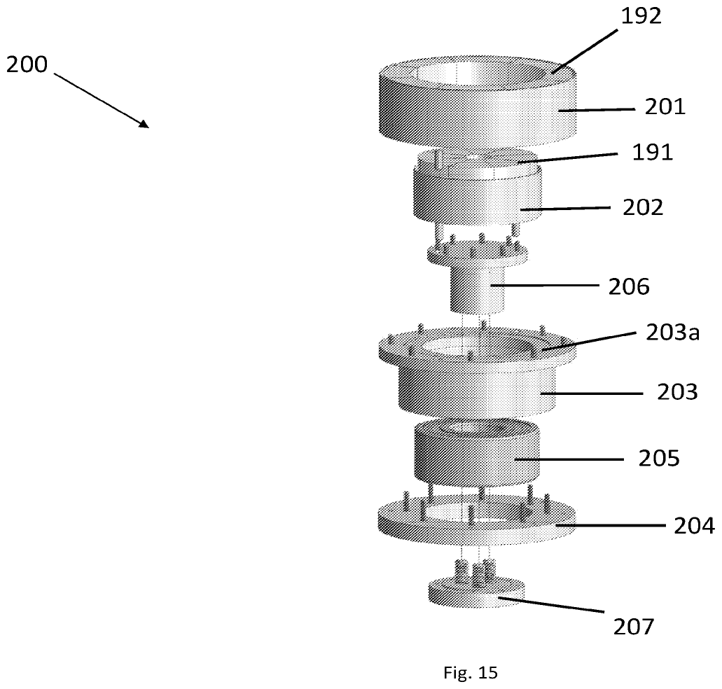

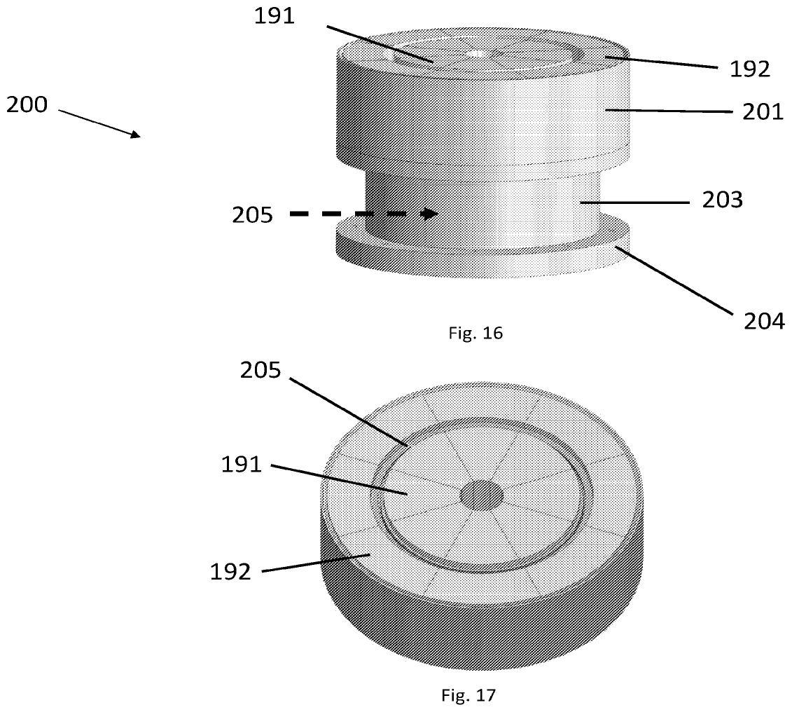

To constrain the cylinders 191 and 192 concentric to each other, some implementations may utilize a bearing structure between the two rings 191 and 192. In some embodiments, this structure may be housed below the two rings (FIG. 15, FIG. 16). In some embodiments, these bearings could be fit within a gap between the two rings (FIG. 17). As shown in FIG. 15, a bearing assembly 200 may include an outer ring 201 partially encasing outer cylinder magnets 192, and an inner ring 202 partially encasing inner cylinder magnets 191, such that inner ring fits within outer cylinder magnets 192. A bearing housing 203 includes a flange 203a providing a surface for the encased inner and outer cylinder magnets (191 and 192) and corresponding rings (201 and 202) to sit atop. Bearing housing 203 is positioned on a retaining plate 204 such that a bearing 205 may be positioned within bearing housing 203. A bearing shaft 206 passes through bearing 205 and engages inner ring 202 and encased inner cylinder magnets 191 at a top end, and further engages a bearing shaft retaining plate 207 at a bottom end. As seen in FIG. 16, the bearing may be positioned such that it is below the inner and outer cylinder magnets 191 and 192, or as seen in FIG. 17, bearing 205 may be positioned within a gap between the inner cylinder magnets 191 and outer cylinder magnets 192.

To actuate the cylinders, a transmission and/or motor mechanism is used such that rings 202 and 201 encasing inner and outer cylinder magnets 191 and 192, respectively, are rotated relative to each other. In some embodiments, the transmission and/or motor mechanism is used to rotate inner and outer cylinder magnets 191 and 192 relative to each other. In some embodiments, the motor or transmission may be geared such that rings 201 and 202 are counterrotated at the specific angular velocities that result in a net zero angular momentum. This transmission could be non-backdrivable such that the torques generated by magnetic interactions between the inner and outer cylinders 191 and 192 do not cause rotation. With these characteristics, power is not required to maintain a specific field strength; only when the field must be varied will the motor be powered and the cylinders rotated. In some embodiments, the transmission and/or motor is configured to move one or both of inner and outer cylinders 191 and 192 up and down, or vertically, along the central axis of both cylinders with respect to each other.

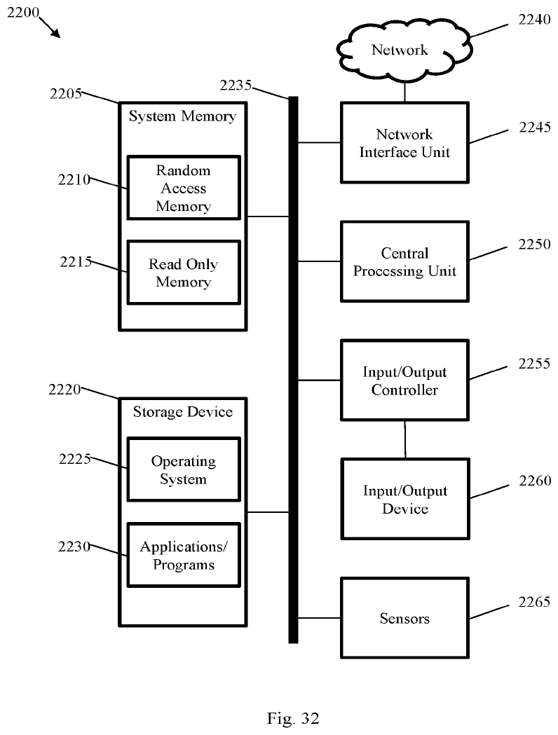

In some embodiments as shown in FIGS. 14-17, the magnetic elements in cylinders 191 and 192 may be rare earth permanent magnets. These elements may be bonded together or mounted within a frame. Relative rotation between the inner and outer rings 202 and 201 would be allowed, through the use of the bearing 205 directly attaching the rings 201 and 202, or each ring being connected to different frame structures. In some embodiments, bearing assembly 200 is communicatively connected to a computing device and mechanically connected to a pyramid receiver for affixing various prostheses. In some embodiments, the computing device of system 100 comprises computer 2200, as shown in FIG. 32.

Accordingly in some embodiments, magnet 120 comprises a magnet array, such as one or more field-adjustable Halbach cylinders. In some embodiments, the magnet array includes permanent magnets. In some embodiments, the magnet array is controlled by changing the relative orientations of multiple permanent magnets within an array. In some embodiments, magnet 120 comprises a MagSwitch® device.

In use, when magnet 120 is an electromagnet that may be powered off (or operating at very low current) during the stance phase of a subject, so as not to create additional stress on the residuum. For an external electromagnet that does not have a permanent magnet core, any current applied to the electromagnet will increase the attractive force between the electromagnet and the implant compared to the zero current condition. During stance phase, any attractive force would only increase compression of the soft tissues because the socket is weight-bearing, so the controller would power off the electromagnet to avoid this. If the external electromagnet has a permanent magnet core, however, a reverse current through the electromagnet would counteract the magnetic field of the core and reduce the attractive force between the electromagnet and implant compared to the zero current condition. During stance phase, the controller could set the electromagnet at a reverse current to limit the additional compression caused by magnet attraction.

Some aspects of the present invention relate to the size, shape and/or features of the implant in relation to size, shape and/or features of the magnet. Generally, the magnetic attraction force is directly proportional to the size of the implant. Material closer to the electromagnet contributes more to the overall attractive force such that surface area of the distal end of the implant has a large influence on the attractive force. The size of the distal end of the implant is sized such that the required magnetic force is achieved. In some embodiments, the diameter of the implant is optimized to the diameter of the magnet to induce the intended magnetic field and magnetic force. In some embodiments, the magnet has a diameter at least as large as the distal face of the implant. For example, in some embodiments the implant has a diameter of about 7 cm and the magnet has a diameter of about 9.8 cm.

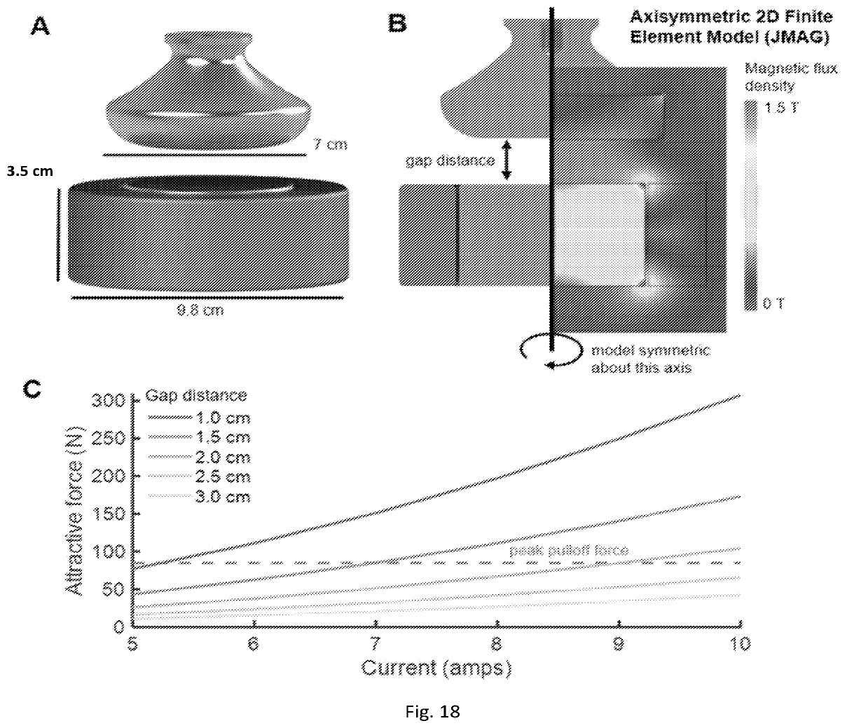

In some embodiments, the ratio of the magnet diameter to the implant diameter is about 1:10, 1:9, 1:8, 1:7, 1:6, 1:5, 1:4, 1:3, 1:2, 1:1, 2:1, 3:1, 4:1, 5:1 and other ratios in between. Some aspects of the present invention relate to the gap distance between the implant and electromagnet. Gap distance is a continuously changing variable. While the aim is to control the gap distance to minimize variations, during operation it may deviate. Now referring to FIG. 18, shown are the approximate dimensions of the implant and electromagnet, as well as results for optimized gap distance. In some embodiments, the system is designed and optimized at a nominal gap distance with a value in the range of 0 cm to 5 cm. In some embodiments, the gap distance is about 0.0 cm, 0.5 cm, 0.75 cm, 1.0 cm, 1.25 cm, 1.5 cm, 1.75 cm, 2.0 cm, 2.25 cm, 2.5 cm, 2.75 cm, 3.00 cm, 3.25 cm, 3.5 cm, 3.75 cm, 4.0 cm, 4.25 cm, 4.5 cm, 4.75 cm, 5.0 cm, 5.25 cm, 5.5 cm, 5.75 cm, or about 6.0 cm.

### Computing Platform

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C #, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

FIG. 32 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 32 depicts an illustrative computer architecture for a computer 2200 for practicing the various embodiments of the invention. The computer architecture shown in FIG. 32 illustrates a conventional personal computer, including a central processing unit 2250 (“CPU”), a system memory 2205, including a random access memory 2210 (“RAM”) and a read-only memory (“ROM”) 2215, and a system bus 2235 that couples the system memory 2205 to the CPU 2250. A basic input/output system containing the basic routines that help to transfer information between elements within the computer, such as during startup, is stored in the ROM 2215. The computer 2200 further includes a storage device 2220 for storing an operating system 2225, application/program 2230, and data.

The storage device 2220 is connected to the CPU 2250 through a storage controller (not shown) connected to the bus 2235. The storage device 2220 and its associated computer-readable media provide non-volatile storage for the computer 2200. Although the description of computer-readable media contained herein refers to a storage device, such as a hard disk or CD-ROM drive, it should be appreciated by those skilled in the art that computer-readable media can be any available media that can be accessed by the computer 2200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 2200 may operate in a networked environment using logical connections to remote computers through a network 2240, such as TCP/IP network such as the Internet or an intranet. The computer 2200 may connect to the network 2240 through a network interface unit 2245 connected to the bus 2235. It should be appreciated that the network interface unit 2245 may also be utilized to connect to other types of networks and remote computer systems.

The computer 2200 may also include an input/output controller 2255 for receiving and processing input from a number of input/output devices 2260, including a keyboard, a mouse, a touchscreen, a camera, a microphone, a controller, a joystick, or other type of input device.

Similarly, the input/output controller 2255 may provide output to a display screen, a printer, a speaker, or other type of output device. The computer 2200 can connect to the input/output device 2260 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 2220 and/or RAM 2210 of the computer 2200, including an operating system 2225 suitable for controlling the operation of a networked computer. The storage device 2220 and RAM 2210 may also store one or more applications/programs 2230. In particular, the storage device 2220 and RAM 2210 may store an application/program 2230 for providing a variety of functionalities to a user. For instance, the application/program 2230 may comprise many types of programs such as a word processing application, a spreadsheet application, a desktop publishing application, a database application, a gaming application, internet browsing application, electronic mail application, messaging application, and the like. According to an embodiment of the present invention, the application/program 2230 comprises a multiple functionality software application for providing word processing functionality, slide presentation functionality, spreadsheet functionality, database functionality and the like.

The computer 2200 in some embodiments can include a variety of sensors 2265 for monitoring the environment surrounding and the environment internal to the computer 2200. These sensors 2265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

In some aspects, the present invention relates to the control of the magnetic suspension system using a computing device. In some embodiments, the computing device of system 100 is computer 2200 of FIG. 32. In some embodiments, system 100 comprises one or more inertial measurement units (IMUs). In some embodiments, system 100 comprises one or more load cells. In some embodiments, system 100 comprises one or more power sources. In some embodiments, system 100 comprises a hip-worn external battery as a power source. In some embodiments, the computing device of system 100 controls the power supplied to the electromagnet. In some embodiments, the computing device of system 100 provides control over the system and the electromagnet based on feedback from sensors, including, but not limited to, various distance sensors: ultrasound, optical reflection, sonomicrometry, linear variable differential transformer (LVDT), and/or various force sensors: load cells, strain gauges, soft-sensors (capacitive, resistive), force sensitive resistor (FSR), pressure sensors, accelerometers, gyrometers, and/or IMUs.

Aspects of the present invention relate to using measurements and/or feedback from various sensors of system 100 to control the magnetic attractive force applied to the ferromagnet implant. In some embodiments, the measurement produced by a load cell within system 100 controls the attractive force applied to the implant. For example, when the load is determined to be compressive, a lower electrical power may be applied to the electromagnet, or a nested magnet may be positioned to have less attractive force. In another example, when the load is determined to be tensile, a higher electrical power may be applied to the electromagnet, or a nested magnet may be positioned as to provide more attractive force. In some embodiments, system 100 and/or power provided to the electromagnet and/or positions of nested magnet array may be controlled by the position of the attached prosthetic limbs. For example, attractive force may be modulated by the percent swing of a prosthetic leg. In another example, attractive force may be modulated by the orientation of a prosthetic arm.

In certain aspects, the present invention relates to sensor measurements relating to the kinematic state of the limb (i.e. accelerometer, gyroscope). In some embodiments, kinematic measurements may be used within a model that describes the socket pulloff force given, but not limited to, limb orientation, speed, acceleration. In some embodiments, information related to the actions of the prosthetic limb (i.e. motor torques, velocities, stability measures of the limb) could also be incorporated into this model. In some embodiments, the output of this model could be used to modulate the magnetic field. In some embodiments, sensors measuring the gap distance, directly (ultrasonic range-finders) or indirectly (hall-effect sensors) could also be used to inform the controller of the current gap distance to compensate for the change in attractive force as a function of gap distance. In some embodiments, signals from electromyography sensors (e.g. information about muscle co-contraction) could be used to inform the commanded magnetic field as well. In some embodiments, sensor measurements could be incorporated into the control of the magnetic field in an open or closed-loop manner with the controlled variable being, but not limited to, a desired gap distance, desired interfacial force, or a desired attraction force between the external magnet and the implant.

In some aspects, the present invention relates to the interoperability of system 100 with other powered and/or sensor-controlled prostheses. In some embodiments, system 100 may form a wired and/or wireless connection with powered prosthetic equipment to share power supply, sensors, controllers and/or metric data. For example, the position of system 100 during a normal gait swing may provide a command to a prosthetic leg wherein the position of the foot and ankle is modulated based on the control command from system 100. In another example, a prosthetic hand may receive control commands from system 100 based on the position and/or orientation of the arm. In some embodiments, system 100 may receive electrical muscle and nerve signals passed through the skin of the subject and apply these signals to control system 100 and/or a prosthetic attachment.

In some aspects, the present invention relates to new socket designs that no longer must cover the majority of the residual limb surface. In some embodiments, new socket designs could incorporate cutouts or slots to house various sensors. In one example, the socket comprises cutouts above specific muscle groups for electromyography (EMG) sensors. This would resolve one of the major issues faced with integrated EMG sensors in sockets, since the enclosing liner or socket shell around the sensor can decrease signal quality due to motion artifacts.

Regarding any potential sensors, one challenge for integration into the electromagnetic attachment system is the high electromagnetic interference (EMI) environment resulting from the changing magnetic field. This is especially important in the case of pressure sensors for sensing forces between the electromagnet and the skin because the electronics cannot be placed directly at this interface. In one embodiment, system 100 may comprise a flexible cavity located at the electromagnet/skin interface that is connected to a pressure sensor outside the high EMI environment via tubing. In some embodiments, the cavity and tubing would be filled with an incompressible fluid to transmit the pressure from the interface to the sensor.

The high EMI environment would also be a problem for any of the source devices for distance sensors. In some embodiments, to measure the gap distance between the electromagnet and implant, one implementation could be modifying the magnet geometry to create an axial hole. In some embodiments, this hole would be filled with a material mimicking the sonic or electromagnetic properties of biological tissue to reduce the material discontinuity between the skin and this filler material. In some embodiments, an ultrasound or LVDT probe located below the electromagnet (and outside the magnetic field) could then measure the gap distance by firing a beam through this filler material and the soft tissue until it is reflected off the bottom surface of the implant.

An additional method of gap distance sensing could involve hall effect sensors. In some embodiments, gap distance sensing could involve hall effect sensors throughout the socket to measure the magnetic field at one or multiple points. In some embodiments, the magnetic field at each point would also be modeled to predict its strength given the current electromagnet output and interaction with the implant geometry for different gap distances. In some embodiments, the gap distance could then be estimated by comparing the readings of the hall effect sensor and model predictions.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

Preliminary experiments and literature review are tailored to demonstrate feasibility of the novel electromagnetic suspension system, with emphasis on the following key requirements: an electromagnet of reasonable size must produce sufficient force to suspend a transfemoral prosthesis across a soft-tissue gap; there must exist ferromagnetic (magnetically “soft”) materials and implant geometries that support longitudinal implantation in the context of a transfemoral residuum; and the dynamics of the electromagnetic force control must support stable fixation and disturbance rejection throughout the gait cycle.

Because these components are crucial to clinical viability of the disclosed approach, each was de-risked through targeted pilot experiments and analysis of the relevant literature.

The first step toward de-risking the disclosed electromagnetic suspension system was to gain an understanding of socket kinematics and interfacial kinetics (forces and moments) during gait. There is a large body of literature describing kinematics and kinetics for persons with above-knee amputation. Many of these studies are focused on measuring pistoning behavior within the socket during characteristic limb movements. Other studies describe transfemoral kinetics in persons with OI; although loading patterns are likely to differ between socket-worn and osseointegrated prostheses, improved socket suspension may enable persons with socket-worn prostheses to adopt the healthier biomechanics seen in conjunction with OI.

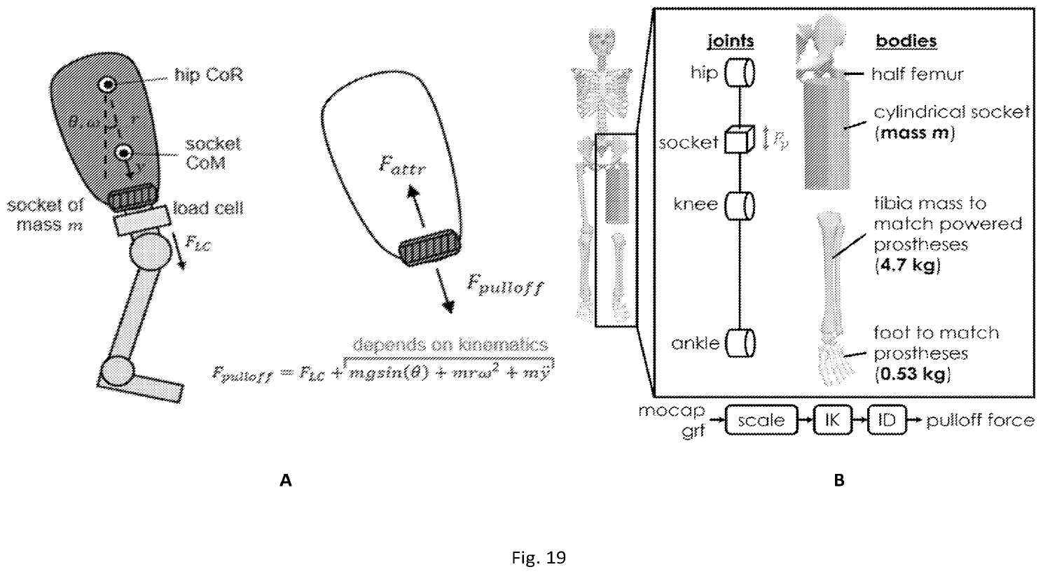

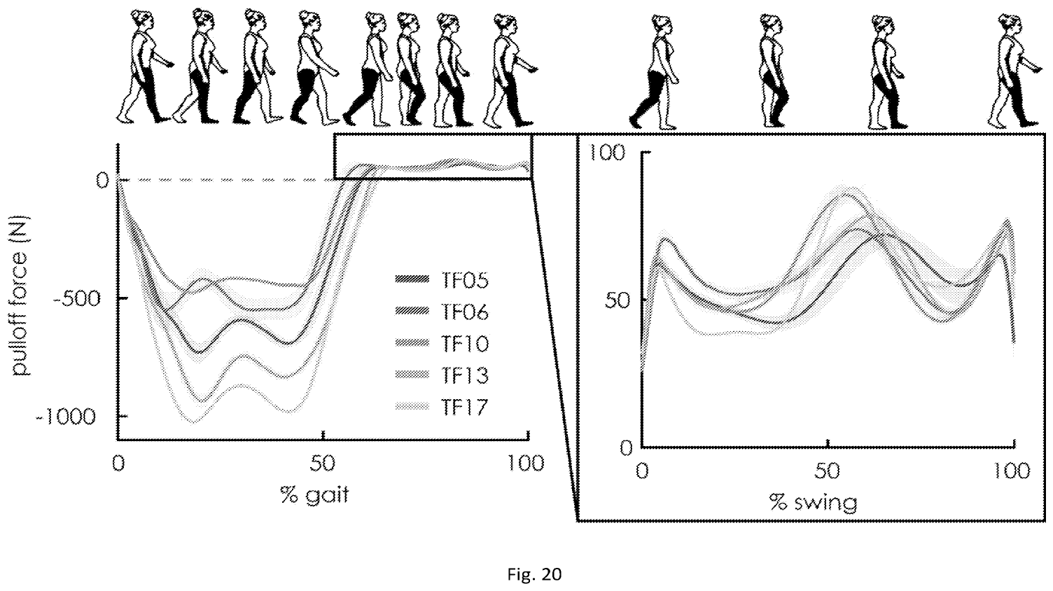

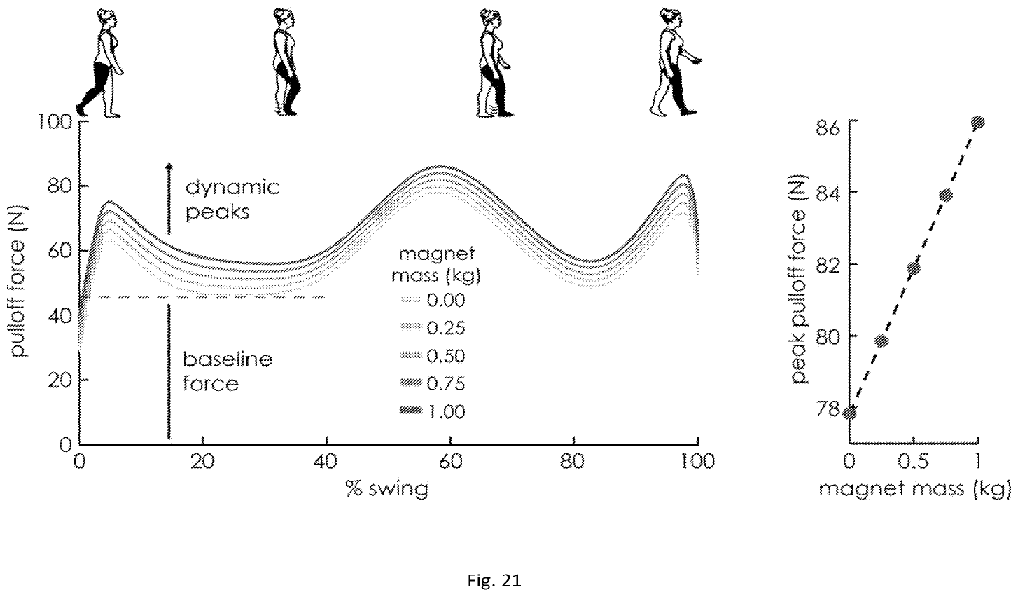

Given that the objective of the disclosed electromagnetic system is to keep the socket affixed to the residuum during dynamic motion, the gravitational, centripetal, and inertial forces acting to pull the socket from the residuum are a crucial consideration (FIG. 19A). These kinematic-dependent forces are especially poignant in light of the electromagnet's added mass within the socket: in addition to supporting the prosthesis, the electromagnetic socket system must also hold itself in place on the residuum. To illustrate this point, and establish a target force profile for the electromagnetic suspension system, employed is an inverse dynamics model (FIG. 19B) to estimate the net axial forces acting to pull prostheses with sockets of different mass from the residual limb during level-ground walking at self-selected speed. The resulting force profiles for different subjects of a published gait dataset of persons with transfemoral amputation are shown in FIG. 20. As a function of socket mass, the inter-subject average pulloff force during swing is shown in FIG. 21. Although socket mass clearly increases pulloff force in a non-trivial way, this additional force will be borne by the residual bone, and not by the soft tissues, which should temper the perception that the prosthesis is “too heavy”. For context, the average biological leg of a 70 kg person weighs approximately 12 kg, such that a 2 kg socket would only account for approximately 17% of the mass of the missing limb.

Another important consideration is the thickness of the skin, which dictates the gap distance across which the electromagnet is expected to act. From preliminary dissections, findings estimate this gap to be 1.5-2 cm, depending on the amount of muscle used for implant coverage.

The next step in de-risking this novel approach is to show that an electromagnet of reasonable size can produce the forces required for robust fixation of an above-knee prosthesis. For a system comprising of a 6 kg prosthetic leg and a 2 kg socket, the peak pulloff force was found to be around 85 N. This peak force sets a feasibility requirement of the proposed system because the electromagnet must be capable of producing this attractive force across a suitable air gap while weighing less than 2 kg.

Toward this end, the study analyzed the disclosed system in electromechanical modeling software (JMAG, JSOL, Tokyo, Japan). To increase computational efficiency in the preliminary analyses, the system was reduced to a two-dimensional axisymmetric representation (FIG. 18A & FIG. 18B). The results of this analysis show that an un-optimized electromagnet with an outer diameter of approximately 10 cm and weighing less than 2 kg is capable of producing the 85 N of attractive force necessary to support its weight, at a gap distance of 1.5 cm, and a 7 amp current (FIG. 18C); the same force over a 2 cm gap requires 9 amps. These peak currents are comparable to those seen in robotic prostheses and can be sourced with conventional battery systems. Thermal output (overheating) is a consideration at these levels of power consumption. It is also important to note that a primary focus of the disclosed work is optimizing the geometry of this electromagnet to maximize force density and minimize heat loss. As such, performance is expected to improve in the final system compared to this preliminary design. However, these early results clearly show that the disclosed system is feasible within the design requirements established in the preliminary biomechanical modeling and cadaver dissections. It is also worth noting that this system may not need to support 100% of the socket pull-off force to add value to the patient. A smaller portion of load suspension (e.g. 70% reduction in pull-off force), in combination with existing suspension technologies, may be sufficient to eliminate pistoning in the presence of daily limb volume changes.

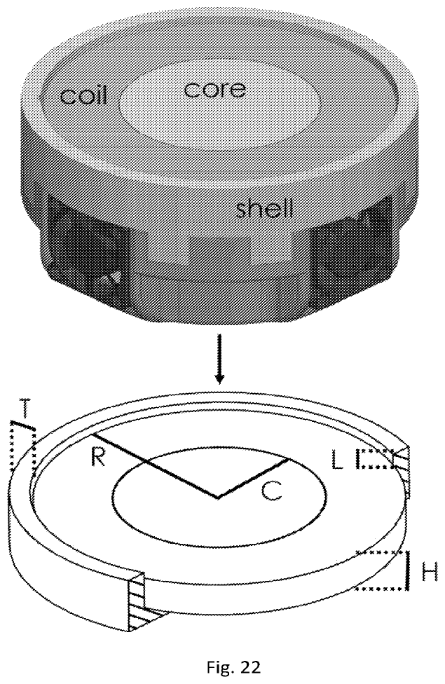

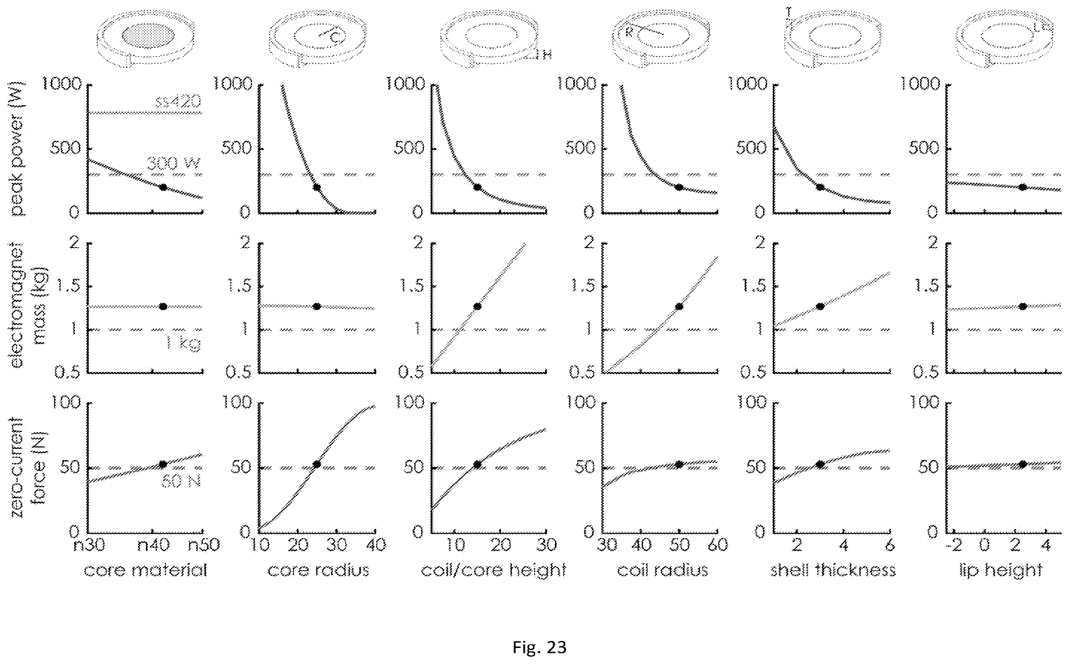

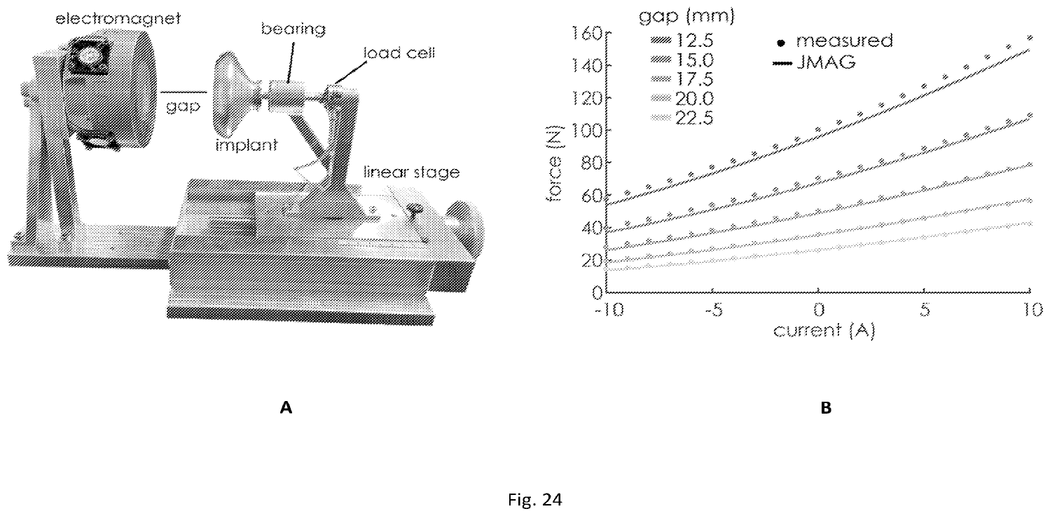

After this preliminary analysis showed that an electromagnet could be used for this application, a generalized electromagnet geometry was created. In this implementation (FIG. 22), the electromagnet consists of a permanent magnet core, coils around the core, and a ferromagnetic shell enclosing the coils. To facilitate design within JMAG, this geometry was then parametrized into the core radius (C), coil radius (R), core/coil height (H), shell thickness (T), and lip height (L). To investigate the effects of each parameter on possible performance metrics for a magnetic attachment system, a sweep on each parameter was performed. As shown in FIG. 23, the peak power required to produce the peak pulloff force during gait decreased exponentially as core radius, coil/core height, coil radius, and shell thickness increased. Higher NdFeB grades also substantially decreased the peak power, albeit in a more linear fashion. Lip height decreased peak power as well, but the effect was relatively small. The mass of the electromagnet increased the most as a function of coil/core height and coil radius, with shell thickness also increasing magnet mass to a lesser degree. Core material, core radius, and lip height had little effect on magnet mass. Zero-current force (attractive force when the electromagnet is powered off) was most affected by the core radius and coil/core height. Increases in NdFeB grade, coil radius, and shell thickness also increased the zero-current force slightly.

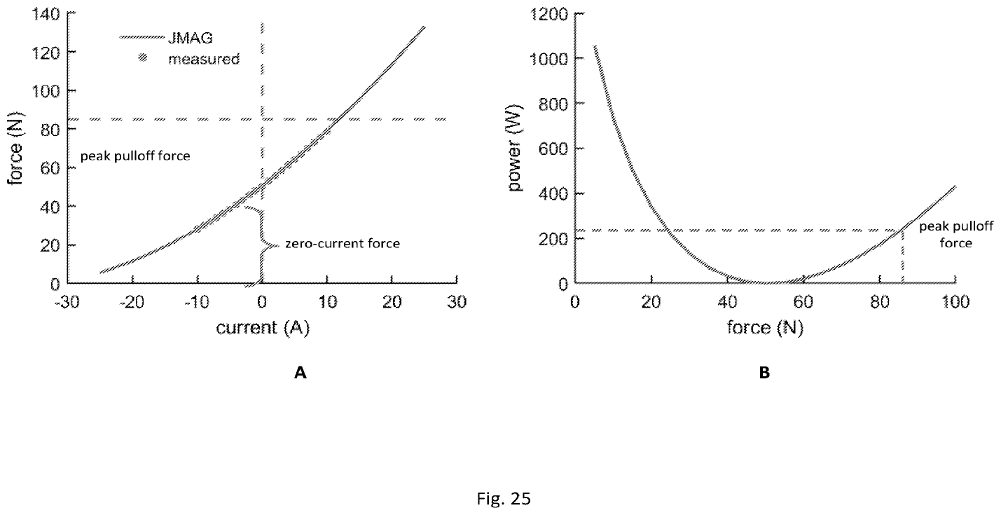

To validate this approach, implemented is a simplified version of the disclosed system on a physical testbench (FIG. 24A). This static testbench is designed to hold a prototype implant at a known distance from an electromagnet, and measure the attractive force produced at different driving currents and gap distances. The electromagnet in this pilot work was the customized electromagnet designed for the disclosed task. The prototype implants were custom-machined from SS 420, which is a likely candidate material for the final system, based on preliminary modeling. The study also compared these results to an equivalent FEA model in JMAG. The results in this pilot study highlight the fidelity of the modeling approach and show that disclosed theory and design is able to produce the forces required to suspend a prosthesis during gait (FIG. 24B).

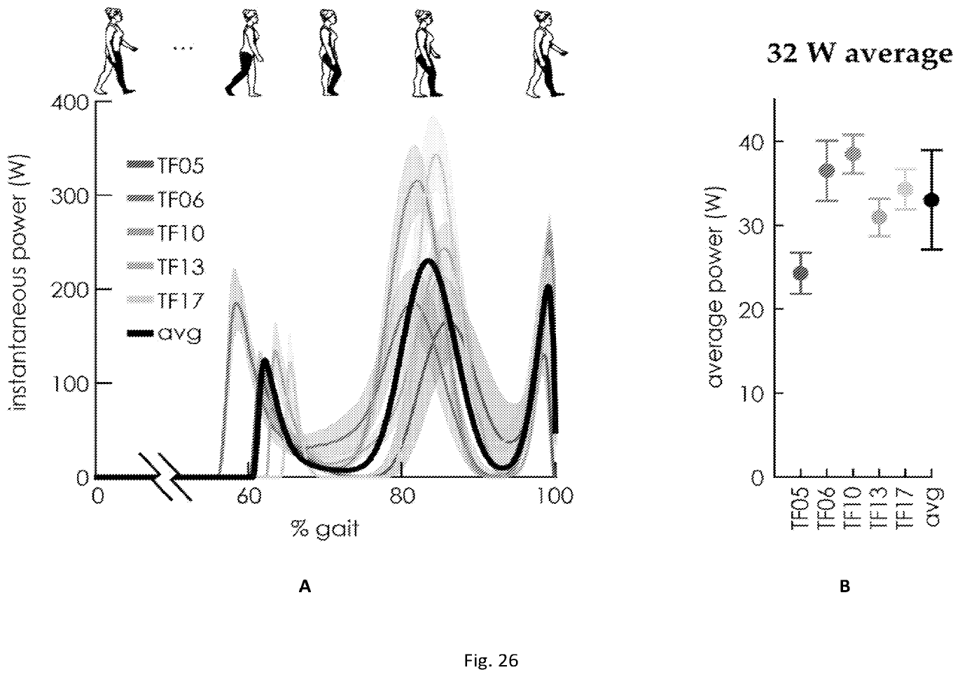

From these preliminary results, embodiments such as those shown in FIG. 9 were designed and optimized in JMAG. Simulating this device with a 17.5 mm gap distance between the top of the electromagnet and the bottom of the implant, the force vs. current (FIG. 25A) and power vs. force (FIG. 25B) curves were calculated. Using the pulloff force profile (FIG. 21), these curves were interpolated to calculate the power required to counteract the pulloff force at each point of the gait cycle (FIG. 26).

Over the entire gait cycle, it is seen that although the peak power of the device is for the inter-subject average pulloff force is around 250 W, the presence of a duty cycle (power is required for around 30% of the whole gait cycle) during gait as the socket is in compression during stance. This results in an average power over the gait cycle of around 32 W, such that a small battery can provide the power needed for 5000 steps.

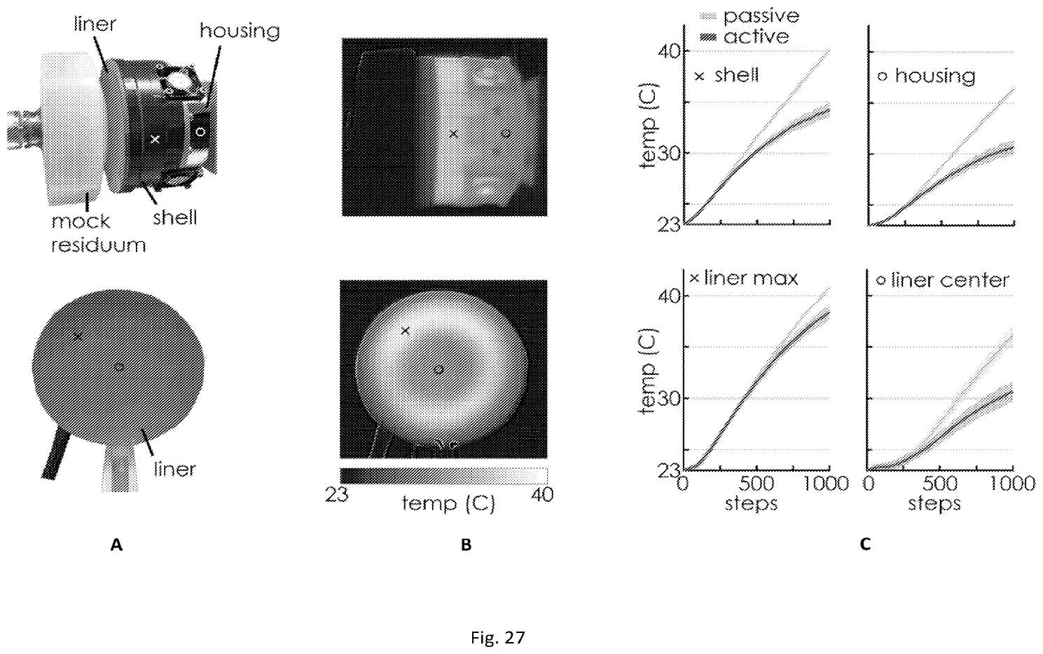

The thermal behavior of the electromagnet during use was experimentally tested using the power profile over the gait cycle (from FIG. 26) and the custom electromagnet. Temperature at locations across the electromagnet were measured using a thermal camera (FIG. 27). At the surface of the liner, which would be in contact with the limb, the temperature increased a maximum of 0.5° C. and 2.3° C. from ambient after 100 and 200 steps, respectively. These increases were similar between active and passive cooling. After 500 and 1000 steps in the active cooling case, the liner temperature had increased by 8.6° C. and 15.4° C. Active cooling consistently reduced the temperatures of all components.

Another important consideration in de-risking the electromagnetic suspension approach is viability of the implant itself. The published work with the greatest relevance comes from prior research into subcutaneous “T-prostheses”. These T-shaped devices, which were implanted under the skin of the residual limb and affixed to the distal end of the transected bone (usually the humerus), were designed to provide mechanical anchoring points for a modified prosthetic socket. Unfortunately, because these devices still relied on direct mechanical load transmission through the soft tissues, early iterations of the device led to the formation of chronic skin lesions that were at risk for implant extrusion. Extrusion occurs when subcutaneous synthetic material abrades the skin and creates an open wound. In current clinical practice, extrusion is a persistent complication in reconstructive facial surgery, remains one of the leading causes of breast implant failure, and is even seen sometimes with pacemakers. Fortunately, gross implant geometry, material selection, and operative approach can help to mitigate these risks. This principle is well-illustrated in one of Inventor Bernthal's tumor cases, in which he implanted a distal femoral endoprosthesis at the time of primary transfemoral amputation to lengthen what would otherwise have been a short residual limb. At one-year post-op, the endoprosthesis sits comfortably below the patient's skin, adding length to her residuum and providing a large load-bearing surface area (FIG. 23A); the patient is now walking comfortably on a prosthetic leg. The success of this case demonstrates that it is possible to place a large, load-bearing metal implant inside the residuum, without leading to skin erosion or extrusion. Newer T-prostheses, in which modifications were made to remove sharp curvatures in the external geometry, have seen similar success in the upper extremities.

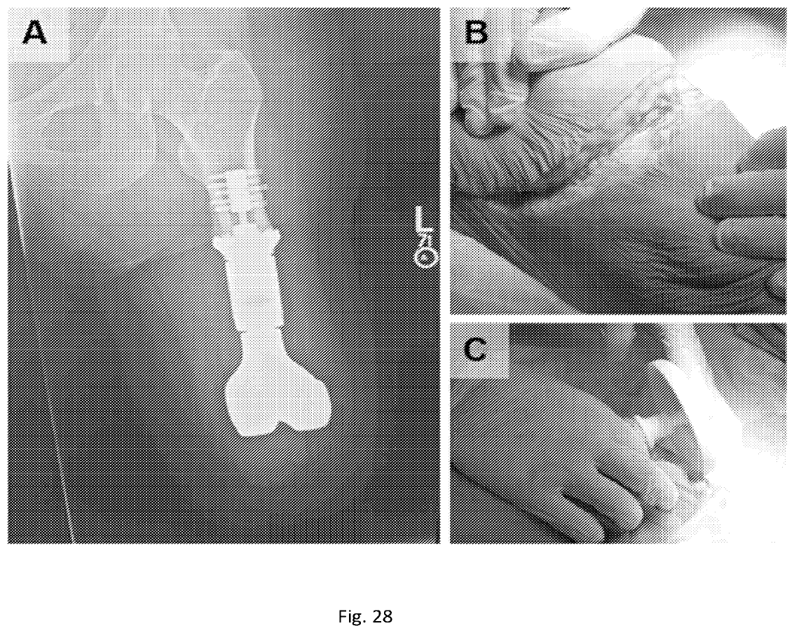

In addition to these prior efforts demonstrating long-term feasibility of the implant in vivo, the acute surgical feasibility of the disclosed implant system was further assessed in a preliminary cadaver dissection. The objectives in this preliminary dissection were to evaluate the thickness of the skin envelope (FIG. 28B) and assess fit of an early implant prototype (FIG. 28C). Also obtained was an initial estimate of the thickness of tissues between implant and electromagnet (1.5-2 cm), and determined ways in which the amputation procedure could be modified to accommodate the implant (e.g. additional bony resection, longer muscle and skin flaps, etc.).

Two potential designs informed by the preliminary dissection are shown in FIG. 6. The top of each implant (hatched surface in FIG. 6) is designed to mate with third-party intramedullary fixation hardware (e.g. Compress from Zimmer Biomet). While the ovular implant has a larger footprint than the circular design, this shape allows a socket to rotationally constrain the attached prosthesis and was also found to provide easier closure of the fishmouth flaps during the surgical procedure.

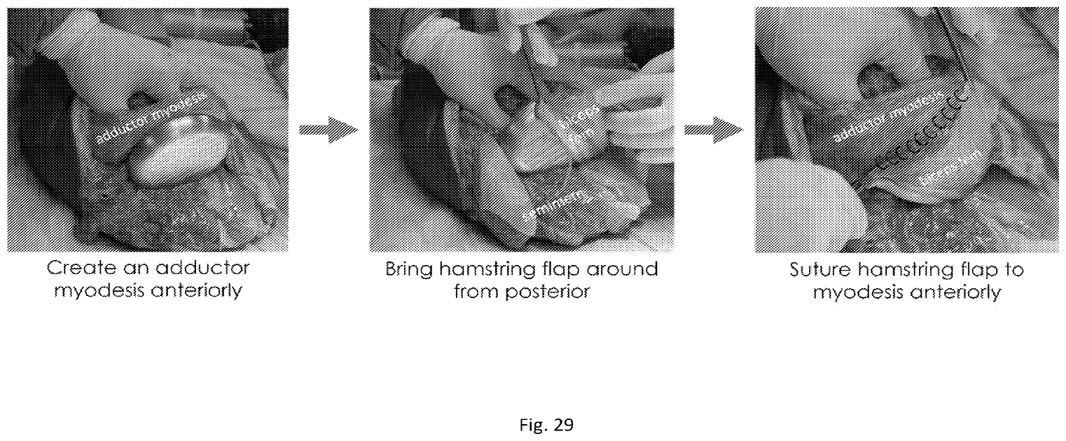

These two designs (FIG. 6) were evaluated in a second dissection to determine the coverage procedure of the implant, estimate the gap distance, and investigate any concerns with closure of the skin over the implant. During this dissection, the ovular implant was found to fit within the required envelope of the residual limb, such that its larger footprint was not an issue. A potential coverage process (FIG. 29) was created such that an adductor myodesis covers the anterior side of the implant, while the hamstring flap is brought around to cover the distal end of the implant and is sutured to the adductor anteriorly. This showed that the implant could be properly covered, and the measured gap distance with this coverage was less than 15 mm.

Material selection for the ferromagnetic implant will also be an important consideration. Although electromagnetic attachment has not been demonstrated in limb prostheses, there is precedent for using implanted ferromagnetic components to improve the longevity of dental prostheses. The majority of these implants are made of ferritic SS (400 series), and many have a Ti—N coating to improve corrosion resistance. The implants are typically coupled with permanent magnets embedded in the external dental prosthesis; the force of attraction between the magnets and the implanted ferromagnetic material acts to hold the prosthesis in place. This prior work serves as a proof of concept for external devices that couple magnetically with bone-anchored subcutaneous implants, albeit under loads that differ substantially from those in a residual limb. Additionally, several studies in the dental field have explored the in vivo viability of coated and uncoated ferromagnetic materials, paving the way for selection of an appropriate material in the disclosed work. On the basis of this prior work, it is expected that a Ti—N-coated 400-series SS will provide sufficient ferromagnetism for prosthetic suspension, and will be biocompatible for long-term implantation. Porous coatings may also be used on some or all of the external surfaces to facilitate soft tissue or bone ingrowth. An example of an implant with a porous/textured coating on one surface in addition to features for suturing tissues to the implant is shown in FIG. 7. Another example of an implant is shown in FIG. 8, where the implant is composed of a ferromagnetic base and a nonferrous cover. The cover could be made of a lightweight material to reduce mass, while also incorporating features for tissue ingrowth or suture attachment.

Note that these disclosed materials would likely render the implant recipient ineligible for MRI. However, in light of other MRI-incompatible medical implants, it is expected that the benefits of improved socket suspension are sufficient to justify this compromise.

A significant body of literature exists describing control strategies for large-gap electromagnetic suspension of ferromagnetic objects. However, it is not currently known how these strategies should be applied to the unique circumstances of prosthetic socket suspension. The objective in the disclosed control system is to regulate the interfacial force within the socket, by manipulating a unidirectional, non-linear attractive force in the presence of unpredictable force disturbances. Reliability, robustness, and stability of this interfacial force control are crucial to the viability of the disclosed system, because drift in either direction can cause significant clinical problems.

To date, the majority of research related to solenoid control has focused on valve regulation or magnetic levitation (“maglev”) transportation. Although this prior work establishes important principles of electromagnetic control, the disclosed research is fundamentally distinct from these applications in a few key ways. First, the armature (in this case, the ferromagnetic implant) does not penetrate the coils of the electromagnet, as it would in a solenoid valve; this limits us to a unidirectional control space (we can attract the ferromagnet, but we can't repel it), and invalidates some of the assumptions inherent to small-gap electromagnetic control. Second, the scale of the present application is drastically smaller than a maglev train; given the nonlinearities inherent to electromagnetic force control, this change in scale has important implications for system stability. Third, because gap distance cannot be measured directly in the disclosed system, and the interfacial medium (in this case, the soft tissue) is a viscoelastic material rather than the air gap that is typical in magnetic levitation, the electromagnetic fixation approach cannot be easily reduced to a position-control problem.

Despite these unique requirements, important insight can still be gained from literature describing benchtop evaluation of large-gap electromagnetic control methodologies. There is a fundamental control challenge inherent in any large-gap electromagnetic suspension, due to significant nonlinearities in the relationship between current, gap-distance, and attractive force. These nonlinearities typically preclude the use of linear control techniques, which are common in conventional small-gap applications, because performance deteriorates rapidly as gap distance moves away from the nominal operating point, leading to potential instabilities. Historically, there have been two key approaches to navigating this non-linearity. The first is gain scheduling, in which the nonlinearities are successively linearized at several discrete operating points, and a linear controller implemented for each point. The primary alternative to the gain scheduling approach is feedback linearization, in which a complete nonlinear model of the electromagnetic field is used by the control system, thereby yielding consistent performance that does not depend on gap distance. Model predictive control has also recently shown promise in improving performance of large-gap electromagnetic suspension systems, although such architectures carry substantial computational overhead.

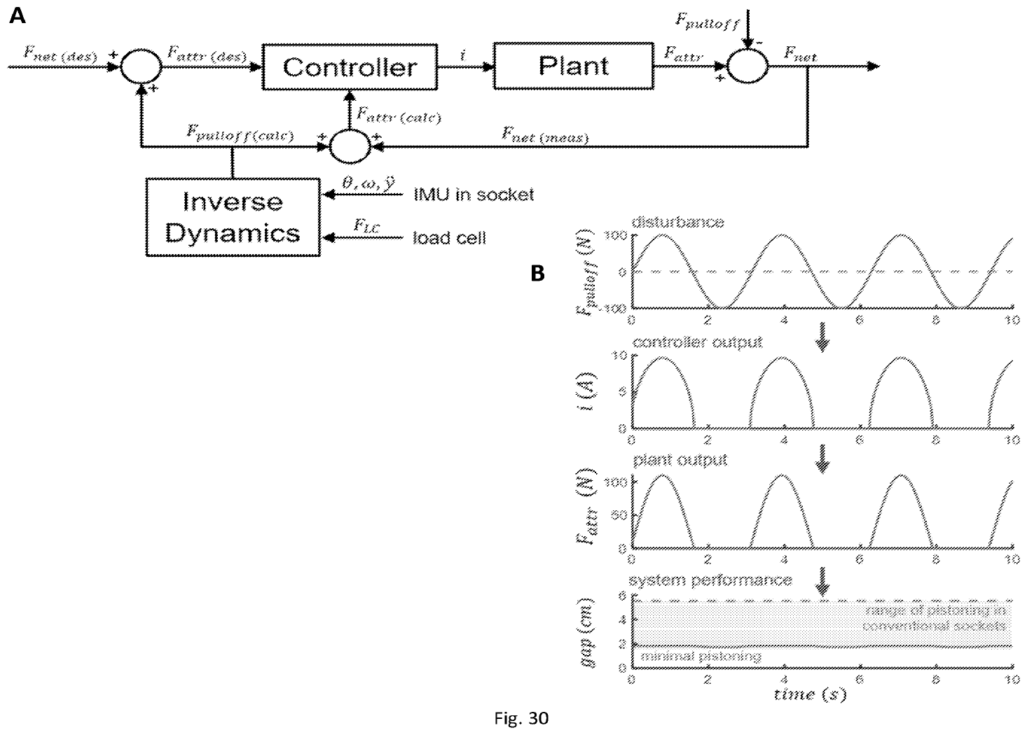

On the basis of this prior work, a preliminary control framework was devised for the electromagnetic control system (FIG. 30A). The disclosed controller seeks to maintain a constant preload within the prosthetic socket, based on feedback from a sensor within the socket wall.

Within this framework, using the results of our preliminary FEA model (FIG. 18), a dynamic simulation of system performance was implemented for a simple proportional-integrative controller, which was tuned manually to minimize pistoning in the context of cyclical disturbance forces. The results of this simulation (FIG. 30B) demonstrate the feasibility of electromagnetic control in creating a stable and robust attachment to the residual limb. Within the general control system, the electromagnet could be controlled based on feedback from many types of sensors, including but not limited to distance sensors (e.g. ultrasound, optical reflection, sonomicrometry, linear variable differential transformer (LVDT)); force sensors (e.g. load cells, strain gauges, soft-sensors (capacitive, resistive), force sensitive resistor (FSR), pressure sensors); or accelerometers/IMUs.

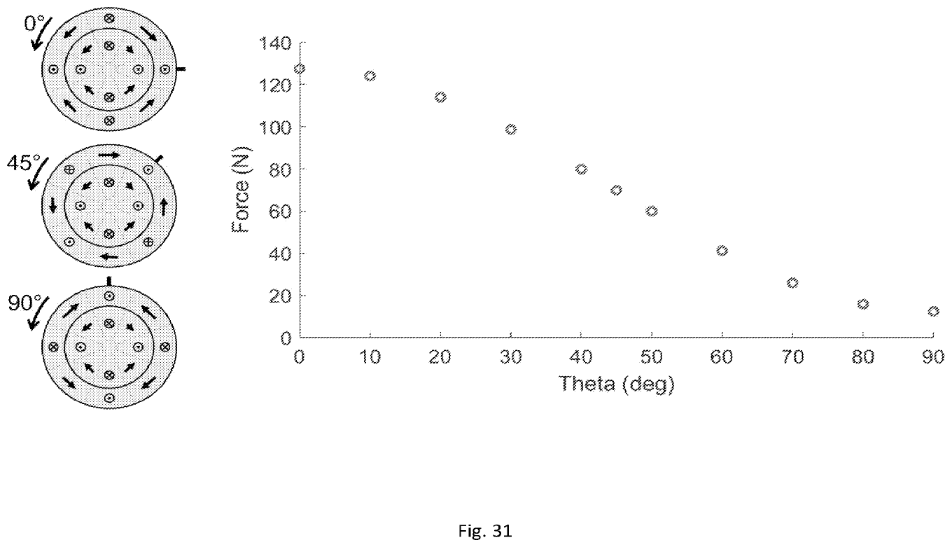

A preliminary model of the device in FIG. 14 was created in the JMAG simulation software, and the feasibility of this device to vary the magnetic field and produce maximum forces within the target range (˜100 N) was tested. In FIG. 31, the attractive force on the implant (in the same setup as the electromagnet implementation) was shown to vary by a considerable amount as the rings are rotated relative to one another through the full cycle (0-90 degrees) of this array implementation (8 segments per ring). The maximum force of around 130 N is also within the range of loads we expect the socket to experience. For applications where a continuous force is required, such as suspending an upper limb prosthesis, this design would be capable of producing these fields with a significant reduction in power compared to an electromagnet design.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A magnetic prosthetic suspension system, comprising:
a magnetic implant configured to be fixedly attached to bone at a target site of a subject;
a socket configured to fit over the target site of the subject; and
a magnet positioned on or within the socket;
wherein the magnet is configured to generate a magnetic attractive force between the magnetic implant and the magnet that suspends the socket at the target site of the subject.

2. The system of claim 1, wherein the magnet is selected from the group consisting of an electromagnet, a permanent magnet, and an electro-permanent magnet.

3. The system of claim 1, wherein the magnet comprises an array of permanent magnets.

4. The system of claim 3, wherein the array of permanent magnets comprises one or more Halbach cylinders.

5. The system of claim 4, wherein the array of permanent magnets comprises an inner cylinder of permanent magnets surrounded by an outer cylinder of permanent magnets, and wherein the array of permanent magnets is configured to produce an adjustable axial flux.

6. The system of claims 3 or 4, further comprising a motor configured to rotate at least one of the magnets.

7. The system of claim 5, further comprising a motor configured to rotate the inner cylinder of permanent magnets relative to the outer cylinder of permanent magnets.

8. The system of claims 6 or 7, further comprising a transmission configured to connect the motor to at least one of the magnets.

9. The system of claim 8, further comprising a power source connected to the socket and configured to power the magnet.

10. The system of claim 9, further comprising a computing device configured to instruct the transmission and motor.

11. The system of claim 1, further comprising an array of sensors positioned in or on the socket comprising at least one of an accelerometer, a gyroscope, a load cell and a pressure sensor.

12. The system of claim 1, wherein the magnet is positioned at the distal end of the socket underneath the implant when the socket is positioned on the target site of the subject.

13. The system of claim 1, further comprising a skin interface layer within the socket and configured to form an interface between the socket and the target site of the subject.

14. The system of claim 1, wherein the socket comprises a receiver configured to engage a prosthetic attachment.

15. The system of claim 1, wherein the magnetic implant comprises a biocompatible coating.

16. The system of claim 1, wherein the magnetic implant comprises tissue in-growth features.

17. The system of claim 1, wherein the magnet forms a cuff around the target site of the subject such that the cuff surrounds at least a portion of the magnetic implant when the socket is positioned on the target site of the subject.

18. A method for magnetic prosthetic suspension, comprising the steps of:
attaching a magnetic implant to a bone at a target site of a subject;
positioning a socket comprising a magnet over the target site; and
generating a magnetic attractive force between the magnet and the magnetic implant, thereby suspending the socket from the target site.

19. The method of claim 18, wherein the ferromagnetic implant is implanted subcutaneously.

20. The method of claim 18, further comprising modulating the magnetic force of the magnet during use.

21. The method of claim 20, wherein the target site is a leg of the subject and the magnetic force is modulated to different values during one or more phases of gait of the subject.

22. The method of claim 18, further comprising positioning a skin interface layer between the socket and the limb.

23. The method of claim 18, further comprising attaching a prosthetic attachment to the socket.

24. The method of claim 20, wherein the modulated magnetic force produces an adjustable axial flux.

25. A permanent magnet assembly, comprising:
a first plurality of permanent magnets forming an inner Halbach array cylinder configured to produce a predominantly axial flux;
a second plurality of permanent magnets forming an outer Halbach array cylinder configured to produce a predominantly axial flux, wherein the inner Halbach array cylinder is nested within the outer Halbach array cylinder such that the inner Halbach array cylinder and the outer Halbach array cylinder are coaxially aligned and a relative rotation between the inner and outer Halbach array cylinders results in an adjustable axial flux.

26. A magnetic prosthetic suspension system, comprising:
a magnetic implant configured to be fixedly attached to bone at a target site of a subject;
a socket configured to fit over the target site of the subject; and
the permanent magnet assembly of claim 25 on or within the socket;
wherein the permanent magnet assembly is configured to generate a magnetic attractive force between the magnetic implant and the permanent magnet assembly that suspends the socket at the target site of the subject.

27. The assembly of claims 25 or 26, further comprising:
a first ring at least partially encasing the first plurality of permanent magnets;
a second ring at least partially encasing the second plurality of permanent magnets;
a bearing housing having a top surface in contact with a bottom surface of each of the first ring and the second ring;
a bearing positioned at least partially within a bearing housing; and
a bearing shaft having a top region and a bottom region, wherein the bearing shaft passes through the bearing and engages the first ring at the top region and is connected to the motor at the bottom region.

28. The assembly of claim 27, further comprising a motor connected to at least one of the inner Halbach array cylinder and outer Halbach array cylinder and configured to rotate the inner Halbach array cylinder and outer Halbach array cylinder relative to each other to produce the adjustable axial flux.

29. The assembly of claim 27, further comprising a transmission.

30. The assembly of claim 27, wherein the motor and transmission are configured to counterrotate the first and second rings at angular velocities that result in a net zero angular momentum.

31. The assembly of claim 27, wherein the motor and transmission are non-backdrivable such that the torques generated by magnetic interactions between the first and second pluralities of permanent magnets do not cause rotation.

32. The assembly of claim 27, wherein the bearing is positioned below the first and second rings.

33. The assembly of claim 27, wherein the bearing is positioned at least partially within a gap between the first and second rings.
